(19) United States
(12) Patent Application Publication
(10) Pub. No.: US 2019/0390151 A1
(43) Pub. Date: Dec. 26, 2019

(54) MEDIUM CHANGING DEVICE AND CULTURE SYSTEM

(71) Applicant: OLYMPUS CORPORATION, Tokyo (JP)

(72) Inventors: Shintaro TAKAHASHI, Tokyo (JP); Koh MOHRI, Tokyo (JP); Tsuyoshi MOCHIZUKI, Tokyo (JP); Asuka NAKAMURA, Kanagawa (JP); Shoichi KANEKO, Tokyo (JP); Shogo USUI, Tokyo (JP); Ikutoshi FUKUSHIMA, Tokyo (JP); Masaru MIZUNAKA, Tokyo (JP)

(73) Assignee: OLYMPUS CORPORATION, Tokyo (JP)

(21) Appl. No.: 16/561,128

(22) Filed: Sep. 5, 2019

Related U.S. Application Data
(63) Continuation of application No. PCT/JP2018/014352, filed on Apr. 4, 2018.

(30) Foreign Application Priority Data
Apr. 7, 2017 (JP) 2017-076469
Jul. 5, 2017 (JP) 2017-131697

(51) Int. Cl. C12M 1/00; C12M 1/12; C12M 1/36

(52) U.S. Cl. CPC C12M 31/00; C12M 29/00; C12M 41/48; C12M 23/26; C12M 41/06; C12M 23/04

(57) ABSTRACT

A medium changing device includes a lid disposed at a position where two or more regions in which a medium may be stored are covered therewith, the regions being disposed adjacent to each other and being open upward; one or more flow-path members each disposed so as to penetrate the lid, and each disposed at a position where one of the regions is bridged to another therewith when the lid is disposed at the position where the regions are covered; and a pump disposed on the other side of the lid and acting on the intermediate sections of the flow-path members, exposed on the other side, so as to cause the medium to flow from the opening at one end toward the opening at the other end of each of the flow-path members.

### CROSS-REFERENCE TO RELATED APPLICATIONS

This is a continuation of International Application PCT/JP2018/014352 which is hereby incorporated by reference herein in its entirety.

This application claims the benefit of Japanese Patent Applications No. 2017-076469 and No. 2017-131697, the content of which are incorporated herein by reference.

### TECHNICAL FIELD

The present invention relates to medium changing devices and culture systems.

### BACKGROUND ART

With the recent advances in stem cell research and regenerative medicine, there has been a demand for preparing large amounts of cells. While being cultured, cells take in ingredients necessary for the growth thereof, such as oxygen and nutrients, and discharge lactic acid and waste products. Thus, a medium becomes degraded when cells are cultured thereon over a long period, which makes it necessary to regularly change the medium. However, medium change is laborious work for the worker.

Furthermore, at the time of medium change, it is necessary to introduce samples into and withdraw the samples from an incubator. This applies stress to the cells, such as temperature and other environmental changes and impacts that occur during transport, which may affect the growth of the cells. Thus, it is preferable to change the medium within the incubator. As an example of a device for changing a medium, a medium changing device disclosed in Patent Literature 1 is known.

### CITATION LIST

### Patent Literature

{PTL 1}

PCT International Publication No. WO 2016/006680

### SUMMARY OF INVENTION

The present invention, in one aspect thereof, provides a medium changing device including a flat-plate-shaped lid disposed at a position where two or more regions in which a medium may be stored are covered therewith, the regions being disposed adjacent to each other and being open upward; one or more flow-path members each disposed so as to penetrate the lid in a thickness direction with openings at either end thereof exposed on one side of the lid and with an intermediate section thereof exposed on the other side of the lid, and each disposed at a position where one of the regions is bridged to another therewith when the lid is disposed at the position where the regions are covered; and a pump that is disposed on the other side of the lid and that acts on the intermediate sections of the flow-path members, exposed on the other side, so as to cause the medium to flow from the opening at one end toward the opening at the other end of each of the flow-path members.

### BRIEF DESCRIPTION OF DRAWINGS

{FIG. 1}

FIG. 1 is an exploded perspective view schematically showing a medium changing device according to a first embodiment of the present invention.

{FIG. 2}

FIG. 2 is an exploded perspective view showing a first modification of the medium changing device in FIG. 1.

{FIG. 3}

FIG. 3 is an exploded perspective view showing a second modification of the medium changing device in FIG. 1.

{FIG. 4}

FIG. 4 is an exploded perspective view showing a third modification of the medium changing device in FIG. 1.

{FIG. 5}

FIG. 5 is an exploded perspective view showing a fourth modification of the medium changing device in FIG. 1.

{FIG. 6}

FIG. 6 is a vertical sectional view showing the medium changing device in FIG. 5.

{FIG. 7}

FIG. 7 is a plan view showing a fifth modification of the medium changing device in FIG. 1.

{FIG. 8}

FIG. 8 is a vertical sectional view showing the medium changing device in FIG. 7.

{FIG. 9}

FIG. 9 is a plan view showing a sixth modification of the medium changing device in FIG. 1.

{FIG. 10}

FIG. 10 is a vertical sectional view schematically showing a medium changing device according to a second embodiment of the present invention.

{FIG. 11}

FIG. 11 is a vertical sectional view schematically showing a medium changing device according to a third embodiment of the present invention.

{FIG. 12A}

FIG. 12A is a vertical sectional view showing a valve in the medium changing device in FIG. 11.

{FIG. 12B}

FIG. 12B is a vertical sectional view showing an operation for opening the valve in FIG. 12A by applying an external force.

{FIG. 12C}

FIG. 12C is a vertical sectional view showing the state where the external force is removed from the state in FIG. 12B.

{FIG. 13}

FIG. 13 is a diagram showing a culture system according to an embodiment of the present invention.

{FIG. 14}

FIG. 14 is a vertical sectional view showing a first modification of the culture system in FIG. 13.

{FIG. 15}

FIG. 15 is a vertical sectional view showing a second modification of the culture system in FIG. 13.

{FIG. 16}

FIG. 16 is a vertical sectional view showing a third modification of the culture system in FIG. 13.

{FIG. 17}

FIG. 17 is a vertical sectional view showing a fourth modification of the culture system in FIG. 13.

{FIG. 18}

FIG. 18 is a vertical sectional view showing a fifth modification of the culture system in FIG. 13.

{FIG. 19}

FIG. 19 is a partial side view showing a seventh modification of the medium changing device in FIG. 1.

{FIG. 20}

FIG. 20 is a partial side view showing an eighth modification of the medium changing device in FIG. 1.

{FIG. 21}

FIG. 21 is a vertical sectional view showing a sixth modification of the culture system in FIG. 13.

{FIG. 22}

FIG. 22 is a vertical sectional view showing a seventh modification of the culture system in FIG. 13.

{FIG. 23}

FIG. 23 is a vertical sectional view showing an eighth modification of the culture system in FIG. 13.

{FIG. 24}

FIG. 24 is a vertical sectional view showing a ninth modification of the culture system in FIG. 13.

{FIG. 25}

FIG. 25 is a vertical sectional view showing a tenth modification of the culture system in FIG. 13.

{FIG. 26}

FIG. 26 is a vertical sectional view showing an eleventh modification of the culture system in FIG. 13.

{FIG. 27}

FIG. 27 is a partial vertical sectional view showing a twelfth modification of the culture system in FIG. 13.

{FIG. 28}

FIG. 28 is a partial vertical sectional view showing a thirteenth modification of the culture system in FIG. 13, in which illumination light is limited by a light blocking member of an observation device.

{FIG. 29A}

FIG. 29A is a plan view showing an example of the light blocking member in FIG. 28, in which the light blocking member has a single circular opening.

{FIG. 29B}

FIG. 29B is a plan view showing an example of the light blocking member in FIG. 28, in which the position of the opening in the radial direction differs from that in FIG. 29A.

{FIG. 29C}

FIG. 29C is a plan view showing an example of the light blocking member in FIG. 28, in which the light blocking member has two openings.

{FIG. 30A}

FIG. 30A is a plan view showing another example of the light blocking member in FIG. 28, in which the light blocking member has a fan-shaped opening.

{FIG. 30B}

FIG. 30B is a plan view showing another example of the light blocking member in FIG. 28, in which the light blocking member has a ring-shaped opening.

{FIG. 31}

FIG. 31 is a partial vertical sectional view showing a fourteenth modification of the culture system in FIG. 13.

{FIG. 32}

FIG. 32 is a partial vertical sectional view showing a fifteenth modification of the culture system in FIG. 13.

{FIG. 33}

FIG. 33 is a partial vertical sectional view showing a sixteenth modification of the culture system in FIG. 13.

{FIG. 34}

FIG. 34 is a partial vertical sectional view showing a seventeenth modification of the culture system in FIG. 13.

{FIG. 35}

FIG. 35 is an illustration showing a modification of the medium changing device in FIG. 1.

{FIG. 36}

FIG. 36 is an illustration showing a modification of the medium changing device in FIG. 1.

{FIG. 37}

FIG. 37 is an illustration showing a modification of the medium changing device in FIG. 1.

{FIG. 38}

FIG. 38 is an illustration showing a modification of the medium changing device in FIG. 1.

{FIG. 39}

FIG. 39 is an illustration showing a modification of the medium changing device in FIG. 1.

{FIG. 40}

FIG. 40 is an illustration showing a modification of the medium changing device in FIG. 1.

{FIG. 41}

FIG. 41 is an illustration showing a medium changing system employing the medium changing device in FIG. 1.

{FIG. 42}

FIG. 42 is an illustration showing a modification of the medium changing system in FIG. 41.

### DESCRIPTION OF EMBODIMENTS

A medium changing device 1 according to a first embodiment of the present invention will be described below with reference to the drawings.

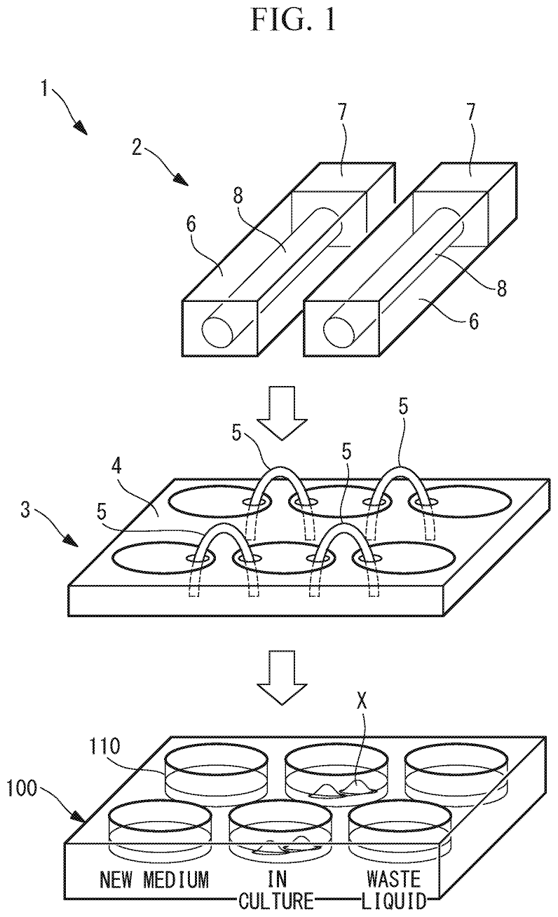

As shown in FIG. 1, the medium changing device 1 according to this embodiment is a device that is used as mounted on a multi-well plate 100 formed by arraying a plurality of wells (regions where media can be stored) 110 at a constant pitch. As shown in FIG. 1, the medium changing device 1 includes power units (pumps) 2 and a liquid transfer unit 3.

The liquid transfer unit 3 includes: a flat-plate-shaped lid member 4 mounted on the multi-well plate 100 at a position where the plurality of wells 110 are covered therewith; and a plurality of flexible tubes (flow-path members) 5 having flexibility and penetrating the lid member 4 in the thickness direction.

When the lid member 4 is mounted on the multi-well plate 100, each of the tubes 5 penetrates the lid member 4 twice in the thickness direction at such positions as to bridge adjacent wells 110 such that both ends thereof are disposed under the lid member 4 and such that the intermediate section thereof is disposed above the lid member 4.

In the example shown in FIG. 1, the multi-well plate 100 includes six wells 110 in two rows and three columns. In the lid member 4, the tubes 5 are disposed at positions such that one tube 5 bridges the well 110 in the first column and the well 110 in the second column and another tube 5 bridges the well 110 in the second column and the well 110 in the third column in the same row. That is, two tubes 5 are disposed per row in the lid member 4.

Each of the power units 2 includes a pump body 6 and a driving unit 7 that drives the pump body 6. The pump body 6 acts on the intermediate section of the tube 5 in the lengthwise direction, exposed above the lid member 4, so as to cause a flow of liquid (medium) in the tube 5. For example, the pump body 6 is a peristaltic pump or the like, which transfers liquid when driven by a method in which the tube 5 is squeezed by a rotor 8 that compresses the tube 5 in the radial direction.

The driving unit 7 is, for example, a motor, and is remotely operated so as to be turned on/off in a wired or wireless manner by a control device, which is not shown. A user may turn on/off the driving unit 7 at a desired timing via the control device, or the control device may turn on/off the driving unit 7 according to a preset program.

The power units 2 are provided such that the power units 2 can be attached to and detached from the lid member 4.

Thus, it is possible to transfer the liquid in the tubes 5 by activating the driving units 7 in a state where the power units 2 are attached to the lid member 4. Furthermore, it is possible to separate the power units 2 from the liquid transfer unit 3 by detaching the power units 2 from the lid member 4. For example, it is possible to configure the liquid transfer unit 3 as a disposable part and to configure the power units 2 as reusable parts.

The operation of the medium changing device 1 according to this embodiment, configured as described above, will be described below.

In order to use the medium changing device 1 according to this embodiment when culturing cells X, media and the cells X are accommodated in the center wells 110 on the individual rows among the six wells 110 in two rows and three columns, new media are accommodated in the wells 110 on one side of the center wells 110, and the wells 110 on the other side are kept empty without any content.

Then, the lid member 4 of the medium changing device 1 according to this embodiment is disposed at a position where the lid member 4 covers the top of the wells 110 accommodating the media and the cells X, and the ends of the tubes 5 penetrating the lid member 4 are disposed inside the individual wells 110. Thus, the tubes 5 are disposed individually at such positions as to bridge adjacent wells 110 among the three wells 110 on each row.

Then, in this state, the power units 2 are attached to the top of the lid member 4. The pump bodies 6 included in the power units 2 are set at the intermediate sections of the tubes 5 in the lengthwise direction, exposed above the lid member 4, whereby the intermediate sections of the tubes 5 are partially compressed in the radial direction. Thus, when the driving units 7 are operated, the compressed portions are moved in the lengthwise direction of the tubes 5 by the rotation of the rotors 8, which makes it possible to cause the internal liquid to flow in one direction.

Cell culture is started after accommodating, in an incubator, the multi-well plate 100 on which the medium changing device 1 according to this embodiment has been installed, as described above.

A user remotely activates the driving units 7 via the control device at a desired timing for performing medium change. First, the pump body 6 installed at the tube 5 between the center well 110 and the empty well 110 adjacent to the center well 110 on each row is driven by the driving unit 7.

Thus, a used medium (waste liquid) that has been used to culture cells X in the center well 110 is sucked into the tube 5 by the power unit 2 and is then discharged into the empty well 110.

Then, the pump body 6 installed at the tube 5 between the center well 110 and the well 110 adjacent to the center well 110 and accommodating a new medium is driven by the driving unit 7. Thus, the new medium stored in the well 110 is sucked into the tube 5 by the power unit 2 and is then supplied to the center well 110.

Thus, with the multi-well plate 100 on which the cells X are being cultured kept accommodated in the incubator, medium change can be performed by discharging the old medium while supplying a new medium. This makes it possible to save the labor of the user involved in medium change. Furthermore, since withdrawals from and reinstallation into the incubator are not involved, an advantage is afforded in that it is possible to avoid stress on the cell X, such as temperature and other environmental changes and impacts that occur during transport, which serves to maintain the integrity of the cells X.

In this case, with the medium changing device 1 according to this embodiment, since the tubes 5 through which media are caused to flow have a short length that is sufficiently long to returns after penetrating the flat-plate-shaped lid member 4 twice in the thickness direction so that the pump bodies 6 can be set, it is possible to reduce the amounts of media that remain in the tubes 5. This makes it possible to reduce the consumption of expensive media and to use general-purpose containers, which results in an advantage that it is possible to reduce the cost of consumables.

Although the multi-well plate 100 having six wells 110 in two rows and three columns is given as an example in this embodiment, the number of wells on a plate that is used, the placement of cells X and unused media, the placement of the tubes 5, etc. may be set, as appropriate, by the user.

Furthermore, although the driving units 7 are remotely operated in the example given above, the driving units 7 may be provided with timers so that the driving units 7 may be turned on/off according to a preset schedule.

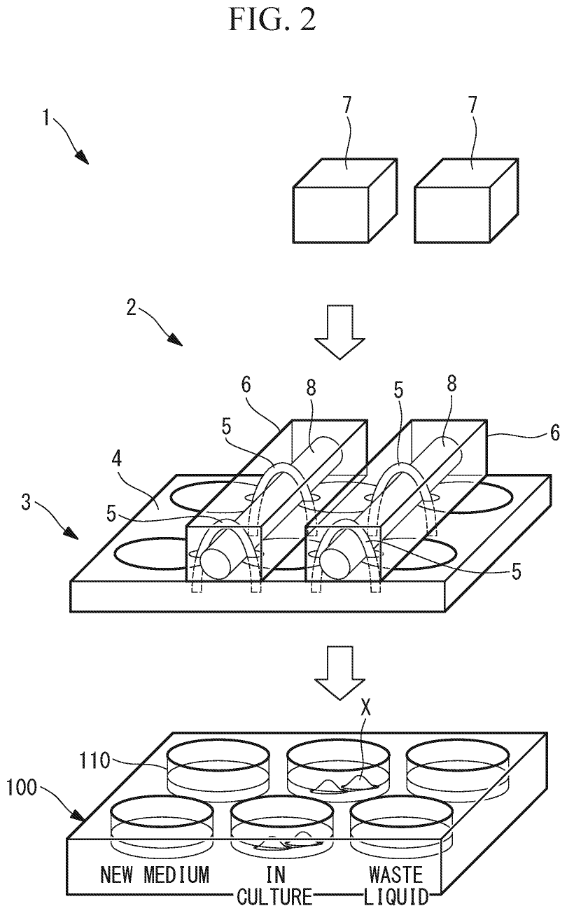

Furthermore, although the liquid transfer unit 3 formed of the lid member 4 and the tubes 5 is configured as a disposable part and the entire power units 2 are reused in this embodiment, alternatively, as shown in FIG. 2, the pump bodies 6 may be attached to the tubes 5, and the driving units 7 may be removably attached to the pump bodies 6. In this case, the pump bodies 6 are also configured as disposable parts. Since it is possible to construct the tubes 5 and the pump bodies 6 in an integrated fashion, it is possible to improve the precision of the amounts of liquid transfer.

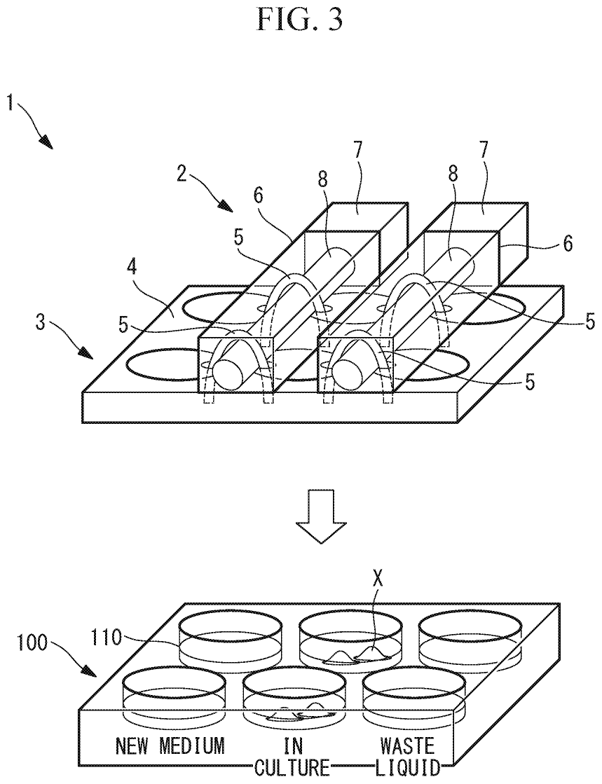

Alternatively, as shown in FIG. 3, the medium changing device 1 may be configured as a disposable device in its entirety. In this case, instead of motors, parts that do not require electric power, such as springs, may be adopted as the driving units 7.

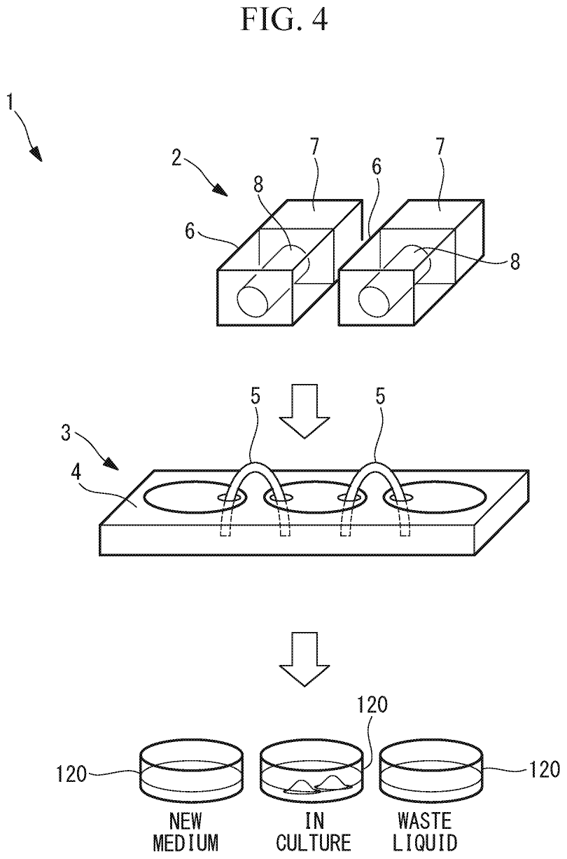

Furthermore, the tubes 5 each bridge two wells 110 on the multi-well plate 100 in this embodiment, alternatively, as shown in FIG. 4, a plurality of culture dishes 120, such as petri dishes, may be used as regions where media can be stored. This makes it possible to allocate a wider culture area compared with the multi-well plate 100.

Furthermore, in this embodiment, the ends of the tubes 5 are disposed at positions close to the bottom faces in the wells 110 at medium inlets (openings) and are disposed at sufficiently high positions separated from the bottom faces in the wells 110 at medium outlets (openings). By disposing the inlets in proximity to the bottom faces, it is possible to reduce the amounts of media remaining in the wells 110 after suction has been performed. Meanwhile, by disposing the outlets away from the bottom faces, the outlets are prevented from contacting the liquid surfaces of the media in the wells 110, which prevents reverse flow of the media.

Furthermore, although the flow-path members are formed of the flexible tubes 5 in this embodiment, there is no limitation thereto, and hard tubes 5 may be adopted. In this case, other kinds of pump bodies 6 should be adopted instead of peristaltic pumps.

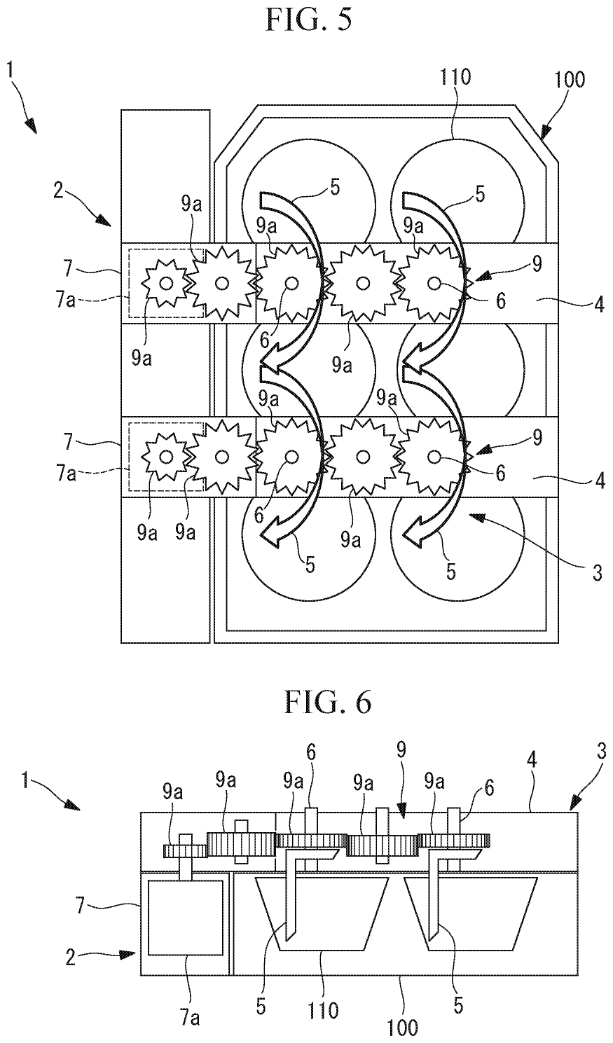

Furthermore, although the driving units 7 such as motors are directly connected to the pump bodies 6 in this embodiment, alternatively, as shown in FIGS. 5 and 6, driving units 7 such as motors 7a may be disposed in proximity to the side faces of the multi-well plate 100 so that the power of the motors 7a can be conveyed to the pump bodies 6 via gear trains 9 formed of a plurality of spur gears 9a. This makes it possible to decrease the entire height in the state where the medium changing device 1 is mounted on the multi-well plate 100.

Furthermore, in this case, it is possible to configure only the tubes 5 and the lid member 4 as disposable parts or to configure the tubes 5, the lid member 4, and the pump bodies 6 as disposable parts. Furthermore, it is also possible to configure portions of the gear trains 9 as disposable parts.

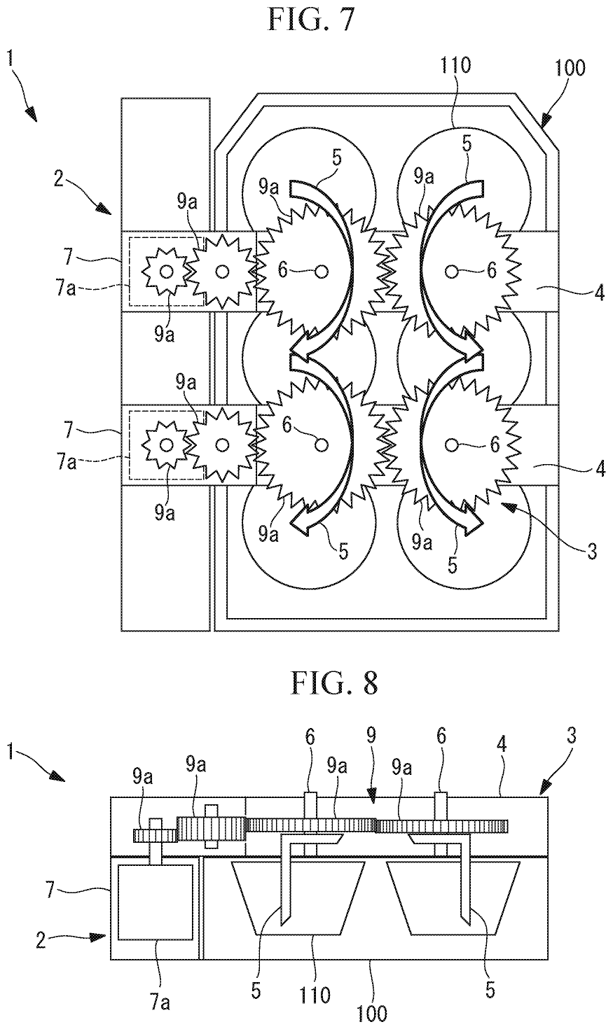

Furthermore, as shown in FIGS. 7 and 8, it is possible to arbitrarily choose the number of spur gears 9a forming each of the gear trains 9.

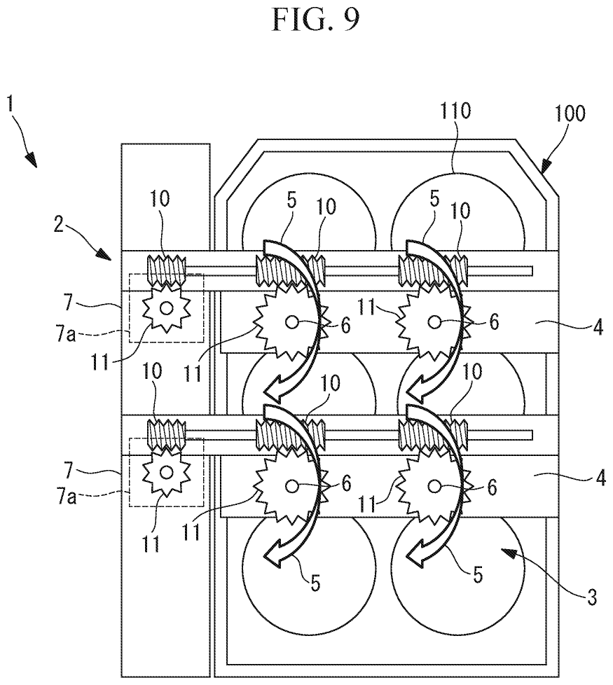

Furthermore, instead of the gear trains 9 formed of the plurality of spur gears 9a, as shown in FIG. 9, the power of the motors 7a may be conveyed by using rack gears 10 and pinion gears 11.

Other modes of this embodiment will be described with reference to the drawings.

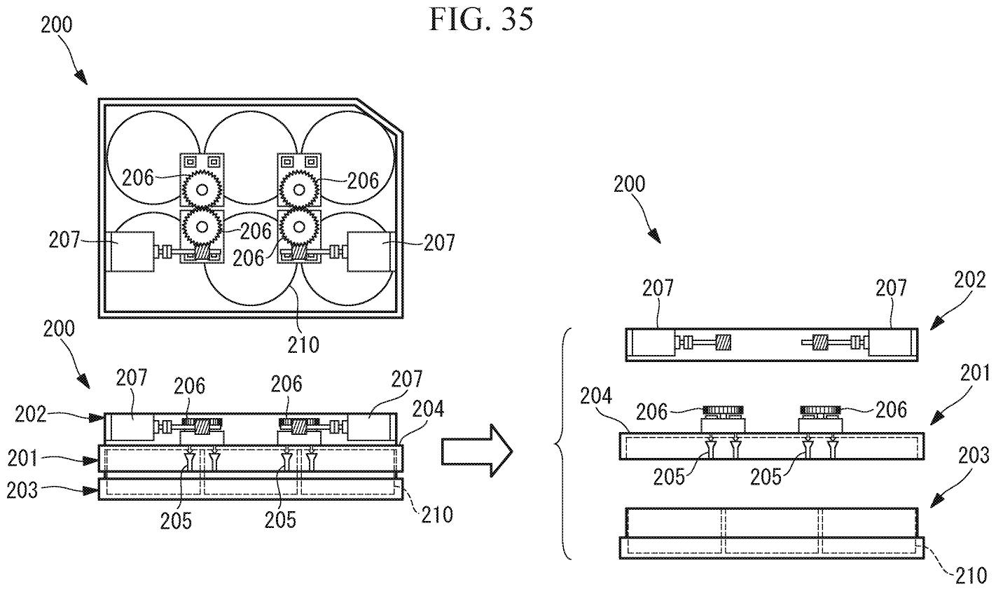

In the mode shown in FIG. 35, a medium changing device 200 includes a liquid transfer unit 201 and a power unit (pump) 202. The liquid transfer unit 201 includes a lid member 204 that is mounted on a multi-well plate 203, tubes (flow-path members) 205 penetrating the lid member 204 in the thickness direction, and pump bodies 206.

The power unit 202 includes driving units 207 that drive the pump bodies 206. By disposing the liquid transfer unit 201 and the power unit 202 in an overlapping fashion, the gears provided on the pump bodies 206 and the gears provided on the driving units 207 are configured to be engaged with each other by their own weight. This configuration simplifies the setup.

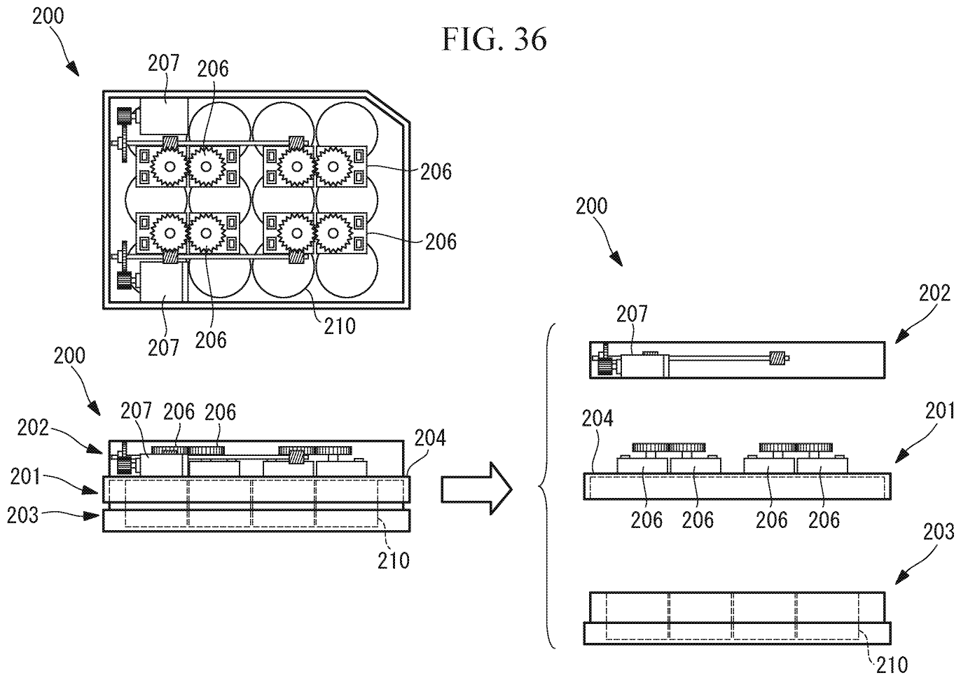

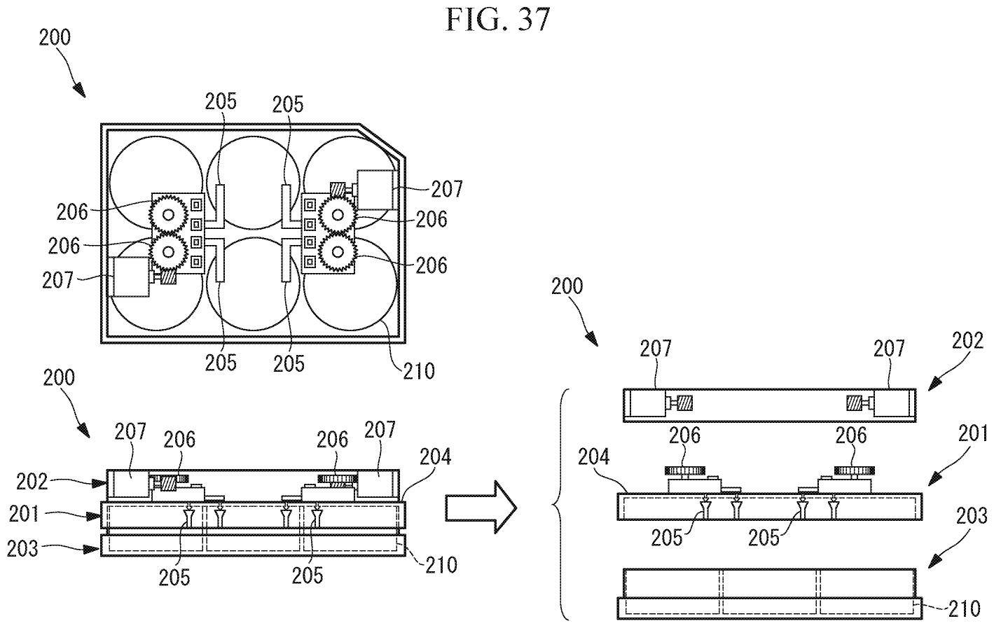

Although FIG. 35 shows an example in which a multi-well plate having six wells is used, the present invention is also applicable to a multi-well plate having twelve wells or a plurality of culture dishes such as petri dishes, irrespective of the number of wells, by optimizing the number and placement of gears for conveying the powers of the driving units 207 to the pump bodies 206, for example, as shown in FIG. 36.

In the mode shown in FIG. 37, a medium changing device 200 includes a liquid transfer unit 201 and a power unit 202. The liquid transfer unit 201 includes a lid member 204 that is mounted on a multi-well plate 203, tubes (flow-path members) 205 penetrating the lid member 204 in the thickness direction, and pump bodies 206. The pump bodies 206 are disposed at positions deviated in the horizontal direction from above the wells 210 in which cells are being cultured.

The power unit 202 includes driving units 207 that drive the pump bodies 206. By disposing the liquid transfer unit 201 and the power unit 202 in an overlapping fashion, the gears provided on the pump bodies 206 and the gears provided on the driving units 207 are configured to be engaged with each other by their own weight. This configuration simplifies the setup. Furthermore, by detaching the power unit 202, it becomes possible to visually check the colors of the media in the wells 210 in which cells are being cultured or to observe the cells by using an inverted microscope.

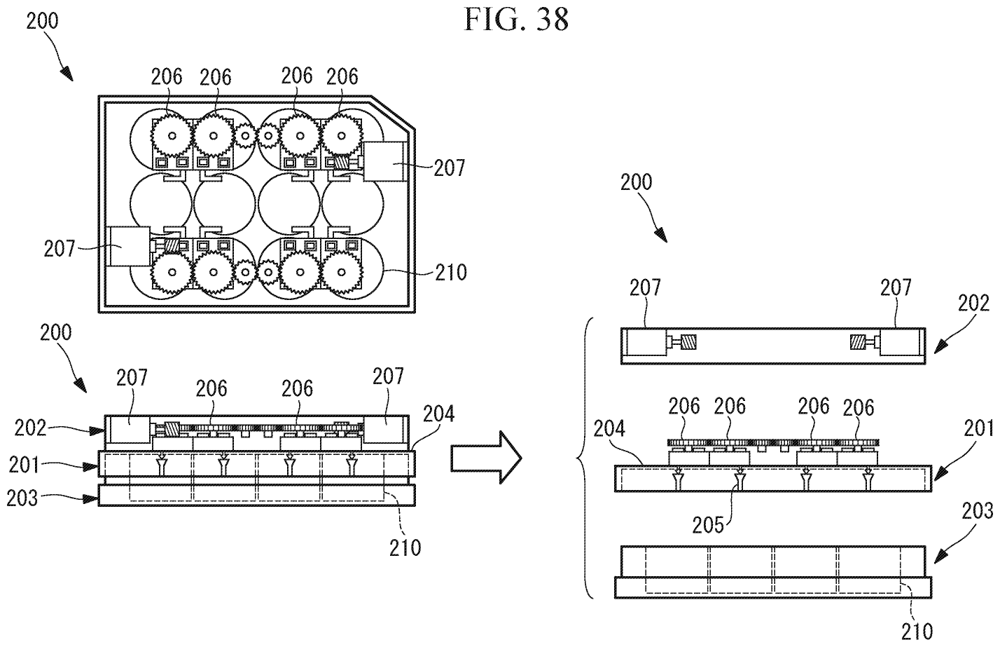

Although FIG. 37 shows an example in which a multi-well plate having six wells is used, the present invention is also applicable to a multi-well plate having twelve wells or a plurality of culture dishes such as petri dishes, irrespective of the number of wells, by optimizing the number and placement of gears for conveying the powers of the driving units 207 to the pump bodies 206, for example, as shown in FIG. 38.

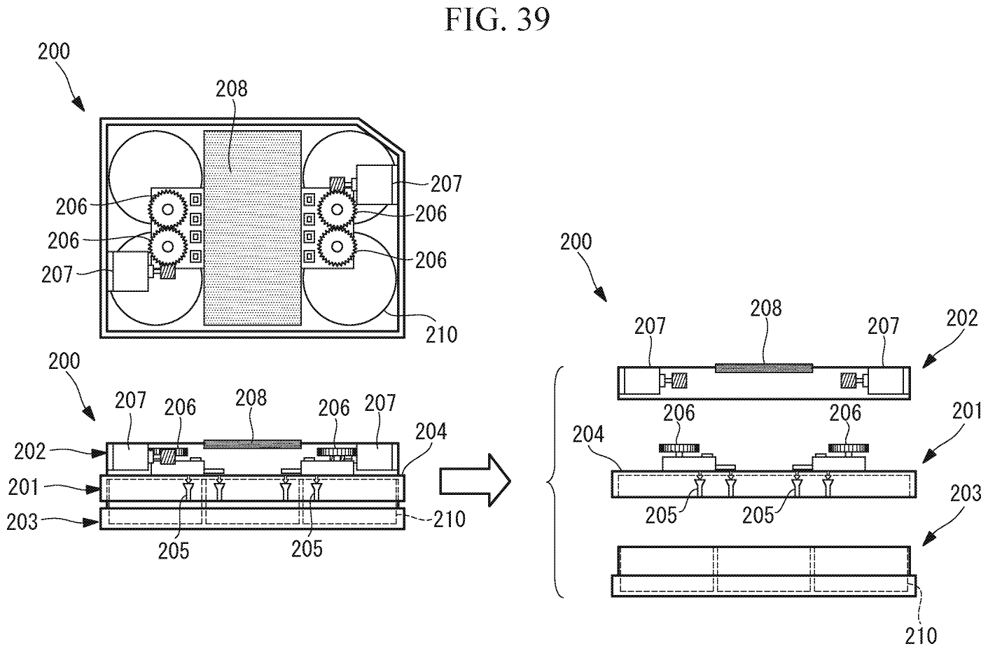

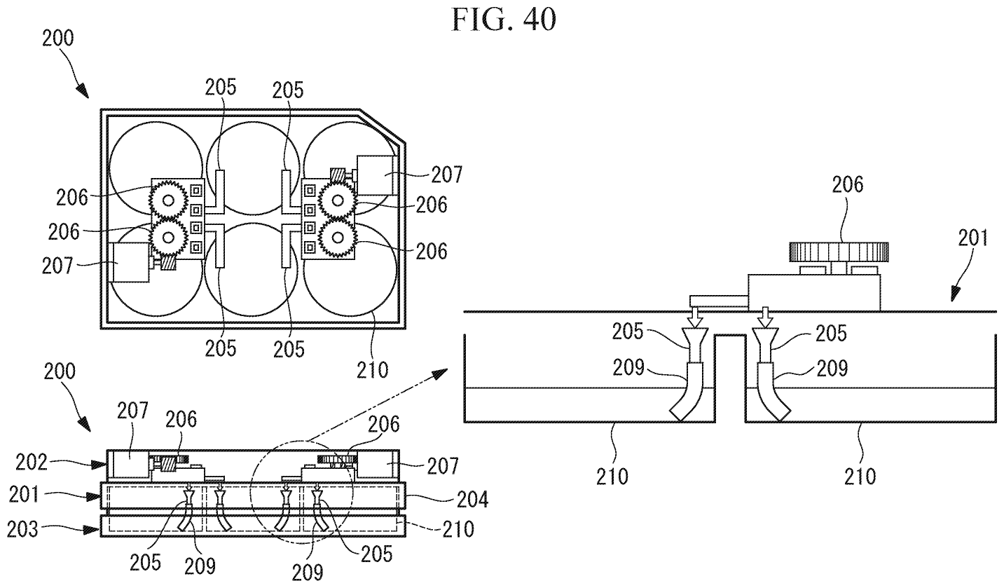

In the mode shown in FIG. 39, a medium changing device 200 includes a liquid transfer unit 201 and a power unit 202. The liquid transfer unit 201 includes a lid member 204 that is mounted on a multi-well plate 203, tubes (flow-path members) 205 penetrating the lid member 204 in the thickness direction, and pump bodies 206. The pump bodies 206 are disposed at positions deviated in the horizontal direction from above the wells 210 in which cells are being cultured.

The power unit 202 includes driving units 207 that drive the pump bodies 206 and an opening or a transparent window 208 made of resin, glass, or the like. The driving units 207 are disposed at positions deviated in the horizontal direction from above the wells 210 in which cells are being cultured. The opening or the transparent window 208 made of resin, glass, or the like is disposed above the wells 210 in which cells are being cultured.

By disposing the liquid transfer unit 201 and the power unit 202 in an overlapping fashion, the gears provided on the pump bodies 206 and the gears provided on the driving units 207 are configured to be engaged with each other by their own weight. This configuration simplifies the setup. Furthermore, without having to detach the power unit 202, it becomes possible to visually check the colors of the media in the wells 210 in which cells are being cultured or to observe the cells by using an inverted microscope.

With the tubes (flow-path members) 205 of the liquid transfer unit 201 in this embodiment, distal ends 209 thereof, which are to be inserted into the wells 210, may be formed of a flexible material, such as silicone tubes. This enables flexible adaptation to containers having wells 210 with different depths. This also applies to second and third embodiments, which will be described below.

Next, a medium changing device 12 according to a second embodiment of the present invention will be described below with reference to the drawings.

In the description of this embodiment, parts that have the same configurations as those of the medium changing device 1 according to the first embodiment described above will be denoted by the same reference signs, and descriptions thereof will be omitted.

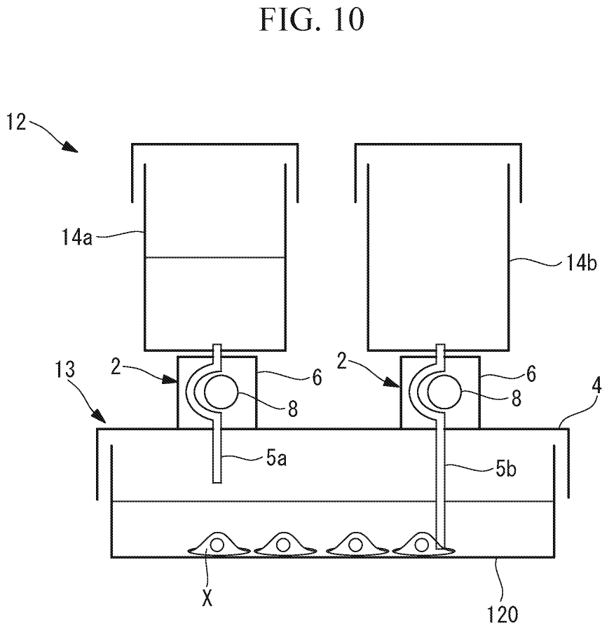

As shown in FIG. 10, the medium changing device 12 according to this embodiment includes power units 2 and a liquid transfer unit 13.

The liquid transfer unit 13 includes a flat-plate-shaped lid member 4 that is disposed at a position where the top opening of a culture dish (a region where a medium can be stored) 120, such as a petri dish, is closed therewith, two tubes (flow-path members) 5a and 5b penetrating the lid member 4 in the thickness direction, and tanks (containers) 14a and 14b disposed above the lid member 4 and connected to the upper ends of the tubes 5a and 5b.

The lower ends of the tubes 5a and 5b are disposed in the culture dish 120 disposed under the lid member 4. The lower end of one tube 5a is disposed at a position close to the bottom face of the lid member 4, and the lower end of the other tube 5b is disposed at a position lower than the lower end of the one tube 5a.

The operation of the medium changing device 12 according to this embodiment, configured as described above, will be described below.

After accommodating cells X and a medium in the culture dish 120, the lid member 4 of the liquid transfer unit 13 is mounted at a position where the top opening of the culture dish 120 is closed therewith. Thus, the lower end of the one tube 5a is disposed at a position higher than the liquid surface of the medium, and the lower end of the other tube 5b is disposed at a position close to the bottom face of the culture dish 120 while being immersed in the medium.

An unused medium is stored in the tank 14a. This tank 14a is connected to the upper end of the one tube 5a, whose lower end is disposed above the liquid surface of the medium. The tank 14b is kept empty. This tank 14b is connected to the upper end of the other tube 5b.

The power units 2 are installed on the top face of the lid member 4. At this time, separate power units 2 are installed individually for the two tubes 5a and 5b.

Then, cell culture is started after accommodating, in an incubator, the culture dish 120 with the medium changing device 12 according to this embodiment installed on the top face thereof.

A user operates the driving units 7 via a control device at a desired timing for performing medium change. First, the driving unit 7 installed for the other tube 5b is activated. The other tube 5b is connected to the empty tank 14b. Since the lower end of the other tube 5b is immersed in the medium, by the operation of the driving unit 7, the medium in the culture dish 120 is sucked, and the sucked medium is discharged into the tank 14a via the other tube 5b. Then, the driving unit 7 installed for the one tube 5a is activated. The one tube 5a is connected to the tank 14a storing a new medium. Thus, the new medium in the tank 14a is supplied into the culture dish 130 via the one tube 5a.

Also, in this embodiment, the tubes 5a and 5b have a short length that is sufficiently long to vertically penetrate the flat-plate-shaped lid member 4 so as to connect the interior of the culture dish 120 under the lid member 4 to the tanks 14a and 14b above the lid member 4. This makes it possible to reduce the amounts of media remaining in the tubes 5a and 5b. This makes it possible to reduce the consumption of expensive media and to use general-purpose containers, which results in an advantage that it is possible to reduce the cost of consumables.

Furthermore, in this embodiment, the lid member 4, the tubes 5a and 5b, and the tanks 14a and 14b may be configured as disposable parts and may be changed each time these parts are used. This simplifies consumable equipment and reduces cost. Furthermore, in addition to the above, the pump bodies 6 may also be configured as disposable parts. Furthermore, in addition to the above, the driving units 7 may also be configured as disposable parts.

Furthermore, although this embodiment is described in the context of the case where the culture dish 120 is used as a region where a medium can be stored, alternatively, a single well 110 of a multi-well plate 100 having a plurality of wells 110 may be adopted.

Furthermore, although the tanks 14a and 14b may be disposed at arbitrary positions, it is possible to make the tube length short by disposing the tanks 14a and 14b above the lid member 4.

Next, a medium changing device 15 according to a third embodiment of the present invention will be described below with reference to the drawings.

In the description of this embodiment, parts that have the same configurations as those of the medium changing device 12 according to the second embodiment described above will be denoted by the same reference signs, and descriptions thereof will be omitted.

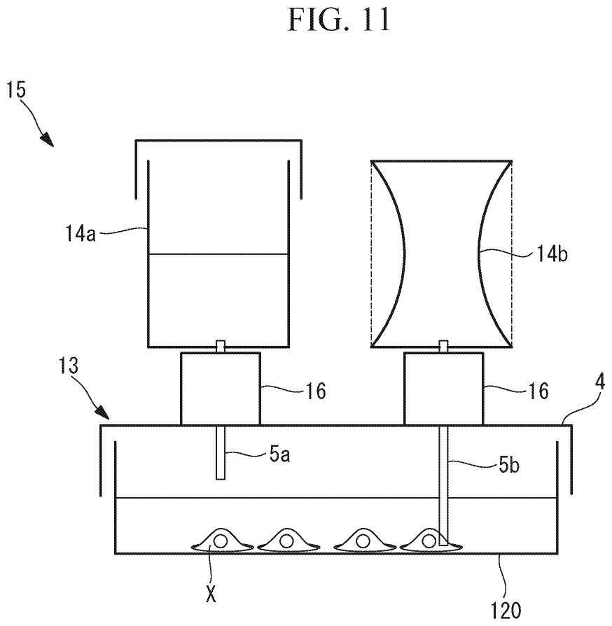

As shown in FIG. 11, the medium changing device 15 according to this embodiment differs from the medium changing device 12 according to the second embodiment in that valves 16 that makes it possible to open and close the tubes 5a and 5b are disposed instead of the power units 2 and in that the pressure in the empty tank 14b is lowered.

In the example shown in FIG. 11, the tank 14b is formed of a material having elasticity. Furthermore, the tank 14b is subjected to elastic deformation to contract the internal volume, and the pressure in the tank 14b is lowered by the elastic restoration ability. Alternatively, the tank 14b may be formed of a hard material, and the pressure in the tank 14b may be lowered by subjecting the interior of the tank 14b to vacuum suction.

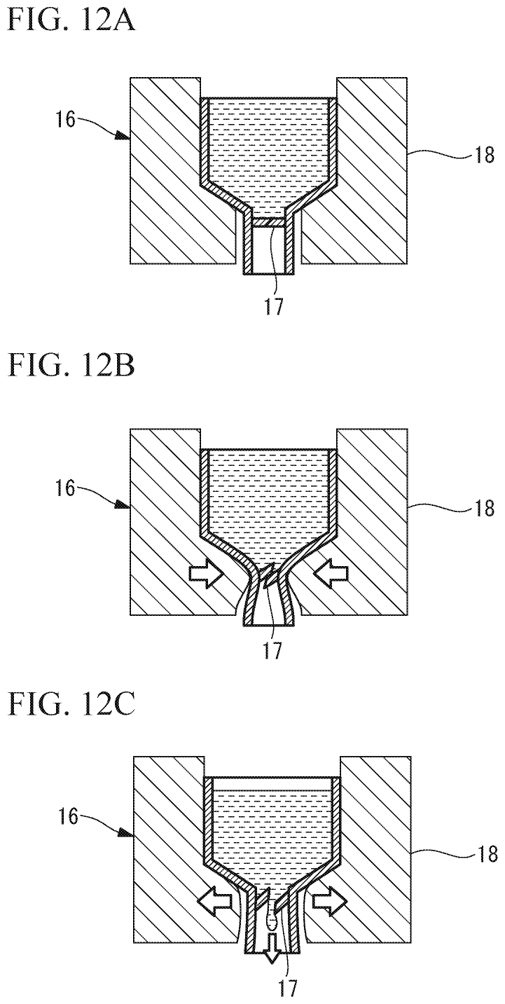

As shown in FIG. 12A, the valves 16 are disposed at intermediate sections, in the lengthwise direction, of the tubes 5a and 5b connecting the culture dish 120 to the tanks 14a and 14b. Each of the valves 16 includes a partition 15 that shuts off the flow path and that is fracturable (breakable) and a pressure applying part 18 for fracturing the partition 17 by applying an external force thereto from the outside of the partition 17. As indicated by arrows in FIG. 12B, the pressure applying part 18 fractures the partition 17 with an external force applied thereto. As shown in FIG. 12C, the pressure applying part 18 is configured so that the flow path will be opened when the applied external force is released after the partition 17 is fractured, which allows the medium to flow.

The operation of the medium changing device 15 according to this embodiment, configured as described above, will be described below.

After accommodating cells X and a medium in the culture dish 120, the lid member 4 of the liquid transfer unit 13 is mounted at a position where the top opening of the culture dish 120 is closed therewith. Thus, the lower end of the one tube 5a is disposed at a position higher than the liquid surface of the medium, and the lower end of the other tube 5b is disposed at a position close to the bottom face of the culture dish 120 while being immersed in the medium.

An unused medium is stored in the tank 14a. This tank 14a is connected to the upper end of the one tube 5a, whose lower end is disposed above the liquid surface of the medium. The tank 14b is kept empty, and the pressure in the tank 14b is lowered by subjecting the tank 14b to elastic deformation. This tank 14b is connected to the upper end of the other tube 5b.

The valves 16 are formed by disposing the pressure applying parts 18 around the partitions 17. The partitions 17 are provided in the tubes 5a and 5b between the individual tanks 14a and 14b and the lid member 4.

Then, cell culture is started after accommodating, in an incubator, the culture dish 120 with the medium changing device 15 according to this embodiment installed on the top face thereof.

A user operates the pressure applying parts via a control device at a desired timing for performing medium change. First, the pressure applying part 18 installed for the other tube 5b is activated to fracture the partition 17 in the other tube 5b. The other tube 5b is connected to the empty tank 14b having a lowered pressure.

Since the lower end of the other tube 5b is immersed in the medium, the medium in the culture dish 120 is sucked into the tank 14b having the lowered pressure and is thus discharged into the tank 14b via the other tube 5b. Then, the pressure applying part 18 installed for the one tube 5a is activated. The one tube 5a is connected to the tank 14a storing a new medium. Thus, the partition 17 in the one tube 5a is fractured, whereby the new medium in the tank 14a is supplied into the culture dish 120 via the one tube 5a by means of gravity.

With this mode, an advantage is afforded in that there is no need for power when sucking and supplying a medium, which allows an even simpler configuration.

Furthermore, since it is possible to reuse only the pressure applying parts while configuring the other parts as disposable parts, consumable equipment is simplified, which serves to reduce cost.

Next, a culture system 20 according to an embodiment of the present invention will be described below with reference to the drawings.

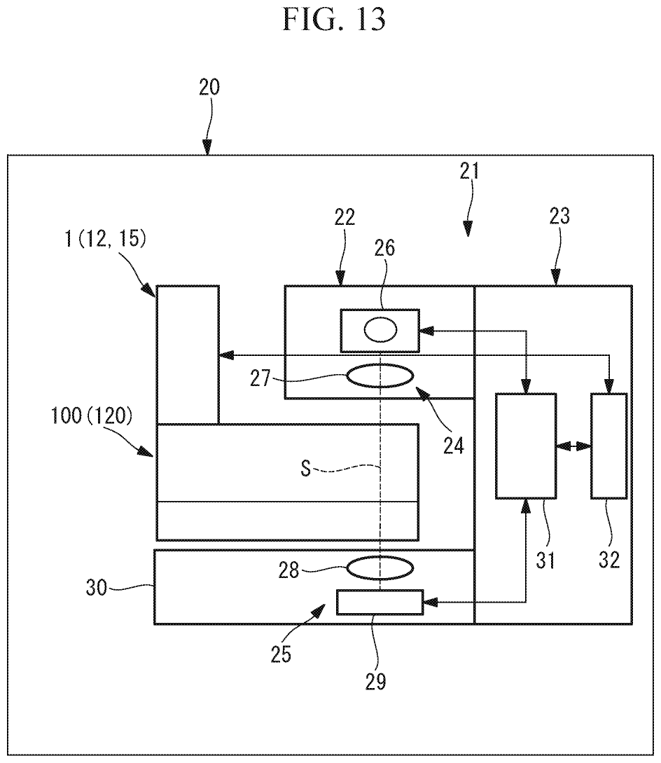

As shown in FIG. 13, the culture system 20 according to this embodiment includes one of the medium changing devices 1, 12, and 15 described above and a culture-state monitoring device 21 for monitoring the state in a region where cells X are being cultured.

The culture-state monitoring device 21 includes an optical data acquisition device 22 and a control device 23.

As shown in FIG. 13, the optical data acquisition device 22 includes: an irradiation optical system 24 that irradiates a medium in the region where the cells X are being cultured with monochrome light; and a measurement optical system 25 that measures the intensity of the monochrome light radiated from the irradiation optical system 24.

The irradiation optical system 24 includes a light source that emits the monochrome light and a collimator lens 27 that substantially collimates the light radiated from the light source 26.

The measurement optical system 25 includes a condenser lens 28 that condenses the monochrome light radiated from the irradiation optical system 24 and a photometer 29 that measures the intensity of the light condensed by the condenser lens 28.

The irradiation optical system 24 and the measurement optical system 25 are disposed so as to face each other in the vertical direction across a culture container, such as the multi-well plate 100 or the culture dish 120, as well as the lid member 4. Hereinafter, the multi-well plate 100 or the culture dish 120 will be referred to as a culture container 100 or 120.

The measurement optical system 25 is accommodated inside a base 30 on which the culture container 100 or 120 is mounted. On the mounting face of the base 30 on which the culture container 100 or 120 is mounted, at least a portion that passes the monochrome light from the irradiation optical system 24 is formed of an optically transparent member.

The control device 23 includes a control unit 31 and a transmitter unit 32. The control unit 31 includes, for example, a central processing unit (CPU) and a memory. The control unit 31 performs ON/OFF control of the light source 26 and computational processing using the light intensity measured by the photometer 29 as the CPU executes various programs stored in the memory. The control unit 31 transmits signals to the medium changing device 1, 12, or 15 via the transmitter unit 32.

The control unit 31 includes, for example, a timer, which is not shown. The control unit 31 is capable of calculating the level of light absorption (absorbance) by the medium over time by periodically activating the light source 26 and the photometer 29. When the level of light absorption by the medium has reached a preset threshold, the control unit 31 transmits a signal to the medium changing device 1, 12, or 15 via the transmitter unit 32. At the medium changing device 1, 12, or 15 that has received the signal, medium change is performed.

The control unit 31 may store the intensity of the monochrome light irradiated from the light source 26 in advance and may compute the level of light absorption by the medium on the basis of the light intensity measured by the photometer 29.

Alternatively, the control unit 31 may determine the timing for transmitting a signal from the transmitter unit 32 on the basis of the intensity of the monochrome light transmitted through the medium, instead of calculating the level of light absorption by the medium.

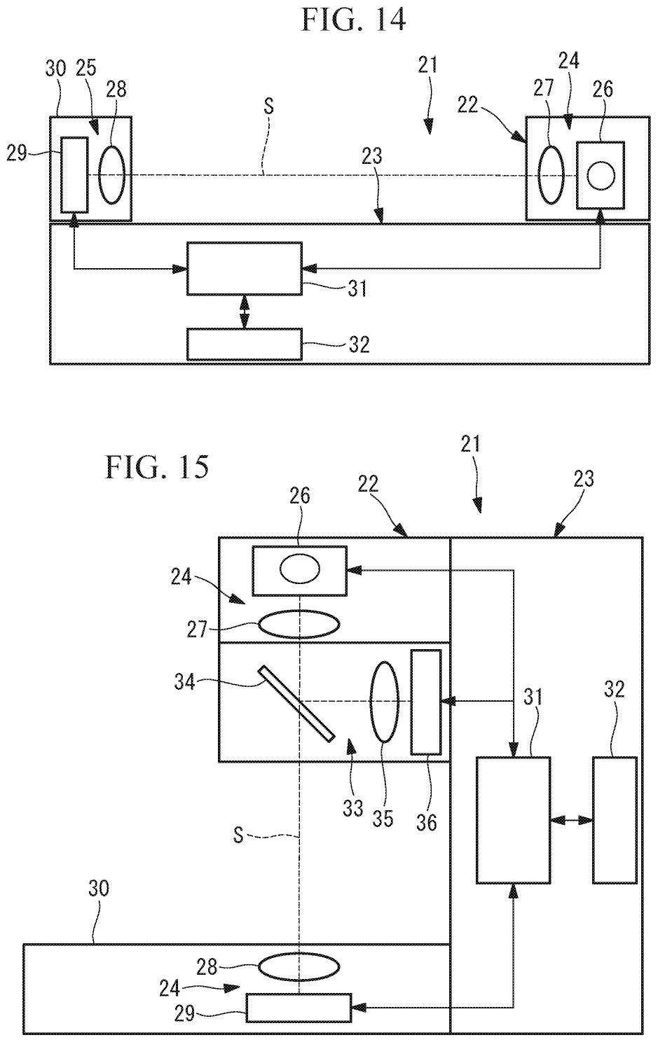

Although the monochrome light is radiated in the vertical direction of the culture container 100 or 120 by transparently forming a portion of the mounting face of the base 30, alternatively, as shown in FIG. 14, the monochrome light may be radiated in the horizontal direction from a lateral side of the culture container 100 or 120. In this case, the culture container 100 or 120 is mounted on the control device 23. This makes it possible to radiate the monochrome light to a position where the monochrome light is transmitted through only a medium where the cells X are absent.

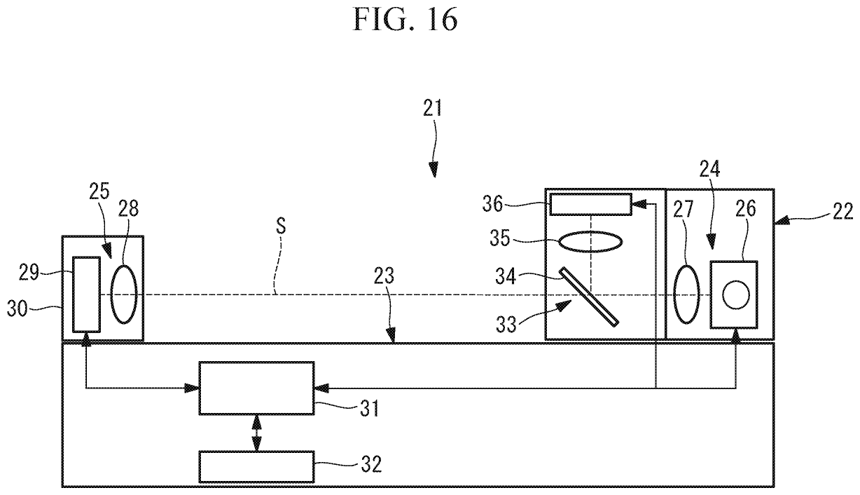

As the culture system 20, alternatively, as shown in FIG. 15 or FIG. 16, a culture system in which an irradiation-light measurement optical system 33 is provided on an optical axis S linking the irradiation optical system 24 and the measurement optical system 25 of the culture system 20 in FIG. 13 or FIG. 14 may be adopted.

The irradiation-light measurement optical system 33 includes a half mirror 34, a condenser lens 35, and a photometer 36.

With this configuration, the monochrome light emitted from the light source 26 is substantially collimated by a collimator lens 27, and the substantially collimated light is split by the half mirror 34. Then, the monochrome light transmitted through the half mirror 34 is introduced to the culture container 100 or 120, while the monochrome light reflected by the half mirror 34 is condensed by the condenser lens 35, and the condensed monochrome light is detected by the photometer 36.

This makes is possible to obtain the difference between the intensity of the monochrome light transmitted through the medium and detected by the photometer 36 and the intensity of the monochrome light detected by the photometer 36 without being transmitted through the medium, and it becomes possible to calculate the absorbance of the monochrome light by the medium on the basis of the difference in light intensity. This makes it possible to determine whether or not the medium is degraded on the spot without having to wait for a change in absorbance over time.

Although the irradiation-light measurement optical system 33 includes the irradiation optical system 24 in this embodiment, a beam splitter that splits light into a reflecting direction and a transmitting direction at a constant ratio may be adopted in place of the half mirror 34. In this case, the control unit 31 should calculate the level of absorption by the medium by performing computation in consideration of the light splitting ratio of the beam splitter. That is, it suffices to include a means for extracting a portion of the incident light, which may be a means for spatially dividing the incident light, such as a mirror that partially reflects only one half of the diameter of the incident light flux.

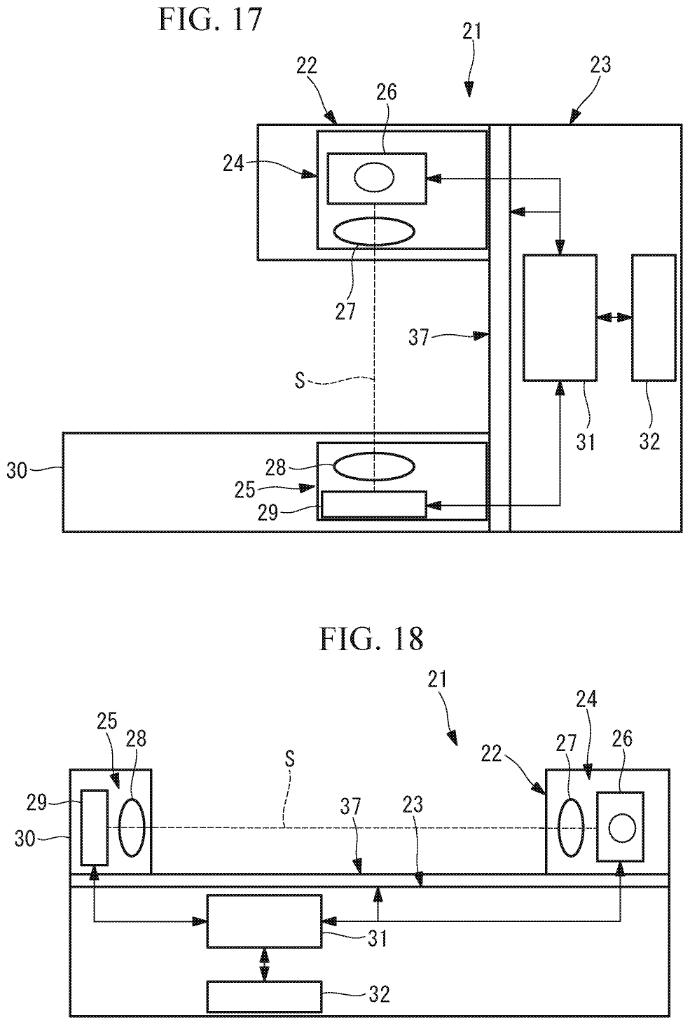

Alternatively, as shown in FIGS. 17 and 18, the culture system 20 according to this embodiment may include a driving means for moving the irradiation optical system 24 and the measurement optical system 25 together relative to the culture container 100 or 120.

The driving means 27 is controlled by the control unit 31 and is capable of moving together the irradiation optical system 24 including the light source 26 and the collimator lens 27 as well as the measurement optical system 25 including the condenser lens 28 and the photometer 29, in the horizontal direction, i.e., the direction perpendicular to the optical axis S linking the irradiation optical system 24 and the measurement optical system 25.

The driving means 37 may include, for example, a linear movement mechanism including a ball screw, which is not shown. In this case, the driving means 37 may move the irradiation optical system 24 and the measurement optical system 25 along a guide rail or the like by transforming rotational motion into linear motion by rotating the ball screw by using a motor or the like. Alternatively, for example, the driving means 37 may include a pulley and a belt, and may move the irradiation optical system 24 and the measurement optical system 25 along a guide rail or the like by transforming rotational motion into linear motion via the belt by applying a rotational force to the pulley by using a motor or the like. The belt may be, for example, a wire or a chain.

With this configuration, it is possible to obtain the intensity of the monochrome light measured without the intervention of the culture container 100 or 120 in a state where the irradiation optical system 24 and the measurement optical system 25 have been moved by the driving means 37 to a position where the optical axis S has deviated from the culture container 100 or 120 and the intensity of the monochrome light measured via the culture container 100 or 120 at a position where the culture container 100 or 120 is disposed on the optical axis S, and to then calculate the level of absorption by the medium by using these light intensities.

Instead of moving the irradiation optical system 24 and the measurement optical system 25 by the driving means 37, the culture container 100 or 120 may be moved. In this case, the base 30 may include a stage for mounting the culture container 100 or 120, and the stage on which the culture container 100 or 120 is mounted may be moved by the driving means 37.

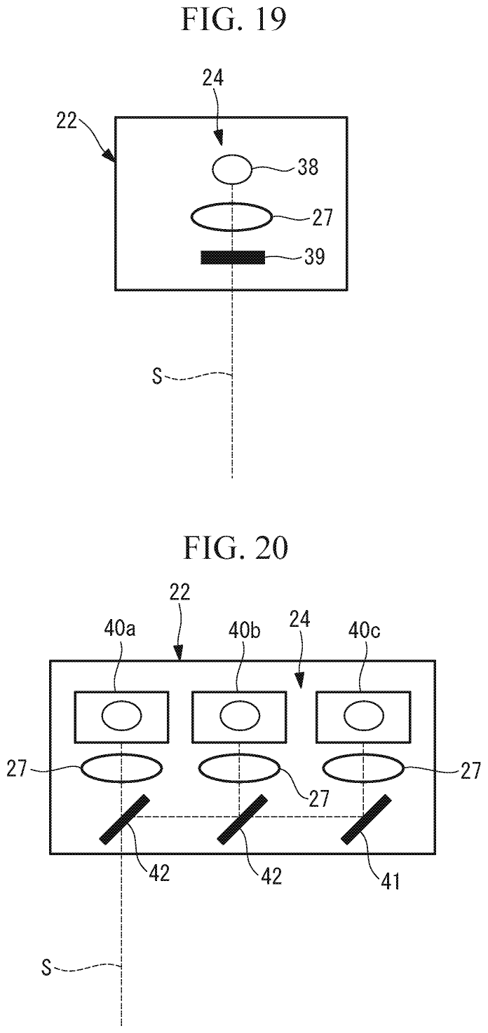

Furthermore, although the mode in which the irradiation optical system 24 includes the light source 26 that emits monochrome light and the collimator lens 27 is given in the embodiment described above, for example, as shown in FIG. 19, a bandpass filter 39 that passes specific wavelengths may be disposed after a white light source 38 and the collimator lens 27.

In this case, the bandpass filter 39 may be configured to be exchangeable so that a bandpass filter 39 that passes desired wavelengths can be inserted into and removed from the optical path.

Alternatively, a plurality of monochrome light sources 40a, 40b, and 40c may be provided, and a desired one of the monochrome light sources 40a, 40b, and 40c may be turned on while performing switching. For example, as shown in FIG. 20, three monochrome light sources 40a, 40b, and 40c that emit light having different wavelengths may be provided, and a mirror 41 and a dichroic mirror 42 may be disposed so as to combine the paths of light from the individual light sources 40a, 40b, and 40c. Then, monochrome light having a desired wavelength may be radiated by turning on a desired one of the monochrome light sources 40a, 40b, and 40c. In this case, the control unit 31 may determine the timing for issuing a signal via the transmitter unit 32 by performing computation, such as obtaining a ratio, on the basis of the levels of absorbance at the plurality of wavelengths.

Furthermore, in the embodiment described above, for example, an LED or an LD may be used as the light source 26 that emits monochrome light, i.e., it is possible to use a light source that emits light having a relatively narrow predetermined wavelength width. Furthermore, desired wavelengths may be extracted by passing the light radiated from the white light source 38 through the narrow-band bandpass filter 39, and the extracted wavelengths may be used for irradiation. Furthermore, it suffices to use a light source that emits light having a wavelength width that allows measurement of the absorbance.

Furthermore, examples of the photometers 29 and 36 include photodiodes (PDs) and photomultiplier tubes (PMTS).

Furthermore, although the case where the collimator lens 27 is included is given as an example, the collimator lens 27 in the irradiation optical system 24 may be omitted depending on the light source 26, 38, or 40a, 40b, and 40c used. Furthermore, the condenser lens 28 in the measurement optical system 25 may be omitted depending on the photometer used.

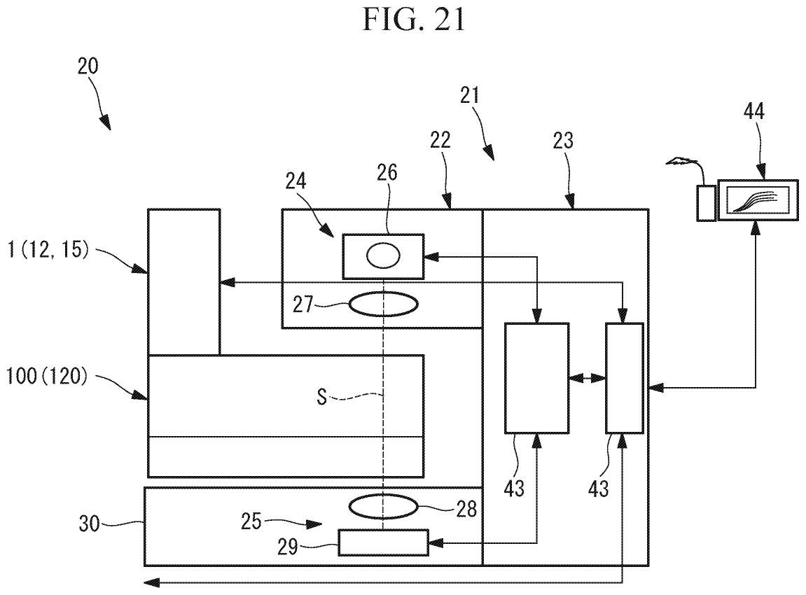

Furthermore, although the control device 23 including the transmitting unit 32 that transmits a signal to the medium changing device 1, 12, or 15 is given as an example, alternatively, as shown in FIG. 21, a control device including a transceiver unit 43 that is capable of transmitting a signal to the medium changing device 1, 12, or 15 and that is capable of receiving a signal from the outside may be adopted. The culture system 20 may include an external control device (control device) 44, and the transceiver unit 43 may transmit signals to and receive signals from the external control device 44, whereby absorbance measurement and medium change are controlled remotely. In this case, the control unit 31 need not include a timer.

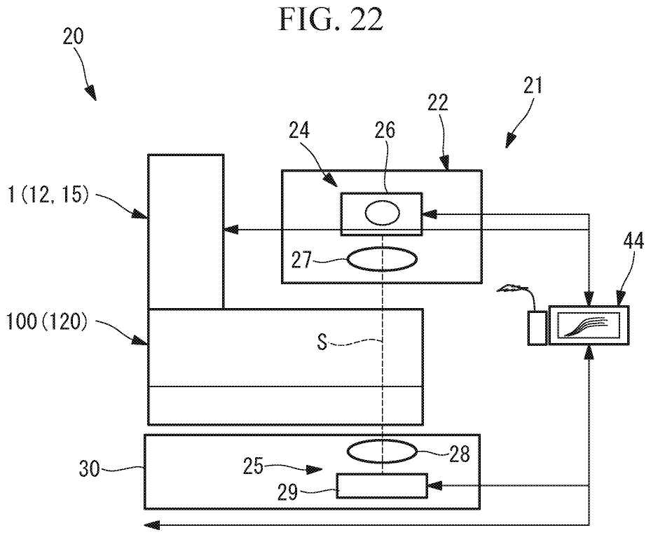

Alternatively, for example, as shown in FIG. 22, the control device 23 may be omitted, and the external control device 44 may directly control the optical data acquisition device 22 and the medium changing device 1, 12, or 15.

An example of the external control device 44 is a personal computer (PC).

For example, the functionality of the external control device 44 may be realized by a PC including a CPU and a memory, which executes a control program stored in the memory. Alternatively, an operator may remotely control absorbance measurement and medium change by operating the PC.

Furthermore, the optical data acquisition device 22 and the culture container 100 or 120 are disposed inside an incubator. The medium changing device 1, 12, or 15 may be disposed inside the incubator, or a portion thereof may be disposed outside the incubator. The control device 23 may be disposed inside the incubator or may be disposed outside the incubator.

Furthermore, although the irradiation optical system 24 irradiates the culture container 100 or 120 with monochrome light from the top face toward the bottom face thereof, the irradiation optical system 24 and the measurement optical system 25 may be disposed in the vertically opposite positions across the culture container 100 or 120 so that the culture container 100 or 120 is irradiated with monochrome light from the bottom face toward the top face thereof.

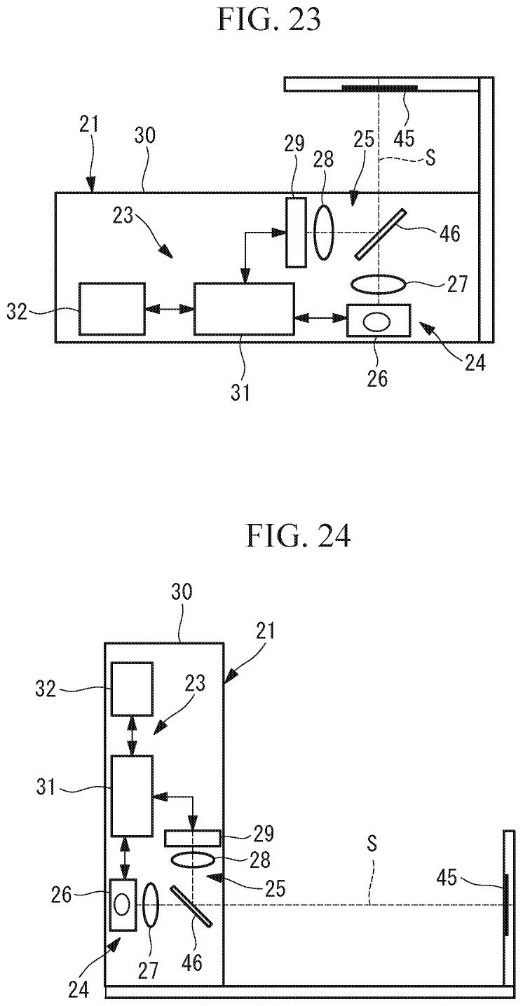

Furthermore, although the irradiation optical system 24 and the measurement optical system 25 are disposed across the culture container 100 or 120, for example, as shown in FIG. 23, the irradiation optical system 24 and the measurement optical system 25 may be disposed on the same side, and a reflecting member 45 may be disposed on the other side across the culture container 100 or 120. In the figure, the reference sign 46 denotes a half mirror that passes the light from the light source 26 and that reflects the light reflected by the reflecting member 45 toward the measurement optical system 25.

In this case, the light radiated from the light source 26 that emits monochrome light is substantially collimated by the collimator lens 27, and one half of the monochrome light, passed through the half mirror 46, irradiates the medium in the culture container 100 or 120. The monochrome light transmitted through the culture container 100 or 120 is reflected by the reflecting member 45 disposed above the culture container 100 or 120 and is again transmitted through the culture container 100 or 120. One half of the light transmitted through the culture container 100 or 120 is reflected by the half mirror 46, the reflected light is condensed by the condenser lens 28, and the intensity of the condensed light is measured by the photometer 29.

The irradiation optical system 24 including the light source 26 and the collimator lens 27, the measurement optical system 25 including the condenser lens 28 and the photometer 29, and the half mirror 46 are accommodated inside the base 30 on which the culture container 100 or 120 is mounted. On the mounting face of the base 30 on which the culture container 100 or 120 is mounted, at least a portion through which the monochrome light passes is formed of an optically transparent member. The control device 23 including the control unit 31 and the transmitting unit 32 may also be accommodated inside the base 30.

Although the reflecting member 45 is attached to the base 30 in an integrated fashion in FIG. 23, the reflecting member 45 may be separate from the base 30. Alternatively, a culture container 100 or 120 having the reflecting member bonded to the top face thereof may be used. The reflecting member 45 is, for example, a mirror.

Alternatively, as shown in FIG. 24, the irradiation optical system 24 including the light source 26 and the collimator lens 27, the measurement optical system 25 including the condenser lens 28 and the photometer 29, and the half mirror 46 may be disposed on a side face of the culture container 100 or 120.

With this mode, it is possible to make the device structure compact, which makes it easier to dispose the device in an incubator. Furthermore, since the monochrome light passes through the medium in the culture container 100 or 120 twice, the level of light absorption by the medium is increased, which makes it possible to improve the sensitivity of detection of changes in the level of light absorption.

Instead of the half mirror 46, a beam splitter that splits light at a constant ratio into a reflecting direction and a transmitting direction may be adopted. In this case, the control unit 31 should calculate the level of light absorption by the medium by performing computation in consideration of the light splitting ratio of the beam splitter. That is, a means for extracting a portion of the incident light may be adopted in place of the half mirror 46, which may be a means for spatially dividing the incident light, such as a mirror that partially reflects only one half of the diameter of the incident light flux.

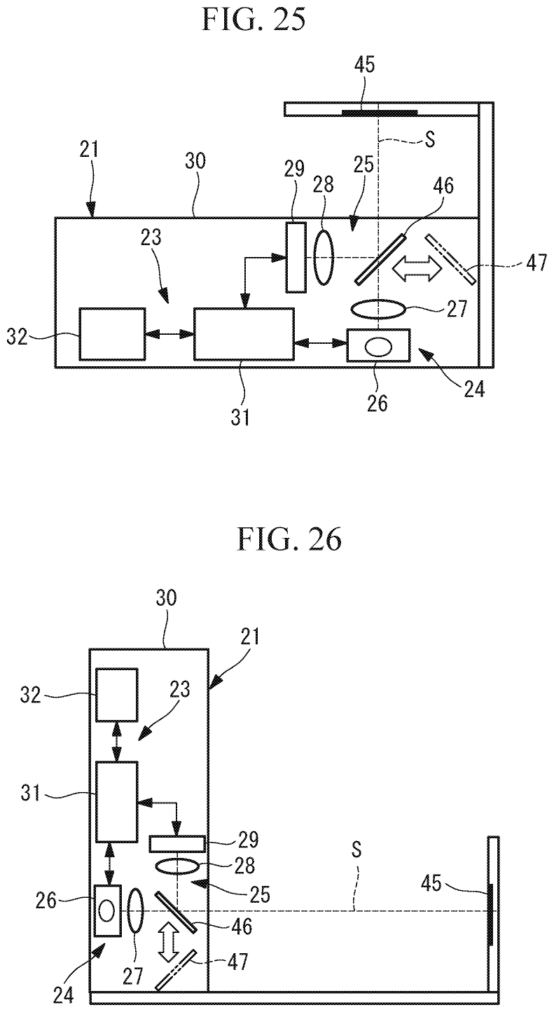

Furthermore, as shown in FIGS. 25 and 26, a half mirror 47 that is disposed so as to be exchangeable with the half mirror 46 may be further included. The half mirror 46 and the half mirror 47 are disposed with inclinations having mutually orthogonal angles. It is possible to dispose the half mirror 46 and the half mirror 47 in an exchangeable manner on the optical axis S by a driving mechanism, which is not shown.

With this configuration, in the state where the half mirror 46 is disposed on the optical path, it is possible to measure the intensity of the monochrome light transmitted through the culture container 100 or 120. Meanwhile, in the state where the half mirror 47 is disposed on the optical path, it is possible to measure the intensity of the monochrome light not transmitted through the culture container 100 or 120. It is possible to calculate the level of light absorption by the medium by the control unit 31 on the basis of the two obtained light intensity data and the light splitting ratios of the half mirrors 46 and 47.

Instead of exchanging the half mirror 46 and the half mirror 47, the angle at which the half mirror 46 is disposed may be rotated by 90° by a driving mechanism, which is not shown.

Alternatively, beam splitters that split light at a constant ratio into a reflecting direction and a transmitting direction may be adopted in place of the half mirrors 46 and 47.

In this case, the control unit 31 should calculate the level of light absorption by the medium by performing computation in consideration of the light splitting ratios of the beam splitters. That is, means for extracting a portion of the incident light may be adopted in place of the half mirrors 46 and 47. The means for extracting a portion of the incident light may be a means for spatially dividing the incident light, such as a mirror for partially reflecting only one half of the diameter of the incident light flux.

Furthermore, the transmission by the transmitter unit 32 may be wired or wireless. Furthermore, the transmission and reception of signals between the external control device 44 and the transceiver unit 43 may be wired or wireless.

Furthermore, examples of the culture containers 100 or 120 include a flask, a petri dish, a culture bag, and a reactor (culture bath).

As the culture system 20 according to this embodiment, a culture system constituted of a first culture system including the medium changing device 1, 12, or 15, the culture container 100 or 120, and the culture-state monitoring device 21 as well as a second culture system including the medium changing device 1, 12, or 15 and the culture container 100 or 120 and not including the culture-state monitoring device 21 may be adopted. In this case, the optical data acquisition device 22 of the first culture system measures the level of light absorption by the medium over time, and the control device 23 may transmit signals to the medium changing devices 1, 12, or 15 of the first culture system and the second culture system when the level of light absorption by the medium has reached a preset threshold.

This makes it possible for the individual medium changing devices 1, 12, or 15 that have received the signals to start discharging and supplying the media by using the signals as triggers.

Furthermore, a plurality of second culture systems may be included. In this case, the control device 23 of the first culture system can transmit signals to the individual medium changing devices 1, 12, or 15 of the first culture system and the individual second culture systems. This makes it possible for the individual medium changing devices 1, 12, or 15 that have received the signals to start discharging and supplying the media by using the signals as triggers.

Furthermore, it is preferable to measure light absorption by phenol red added to the medium. Since phenol red has absorption peaks in the vicinity of 430 nm and in the vicinity of 560 nm, it is preferable to use monochrome light having wavelengths in the vicinities of these wavelengths.

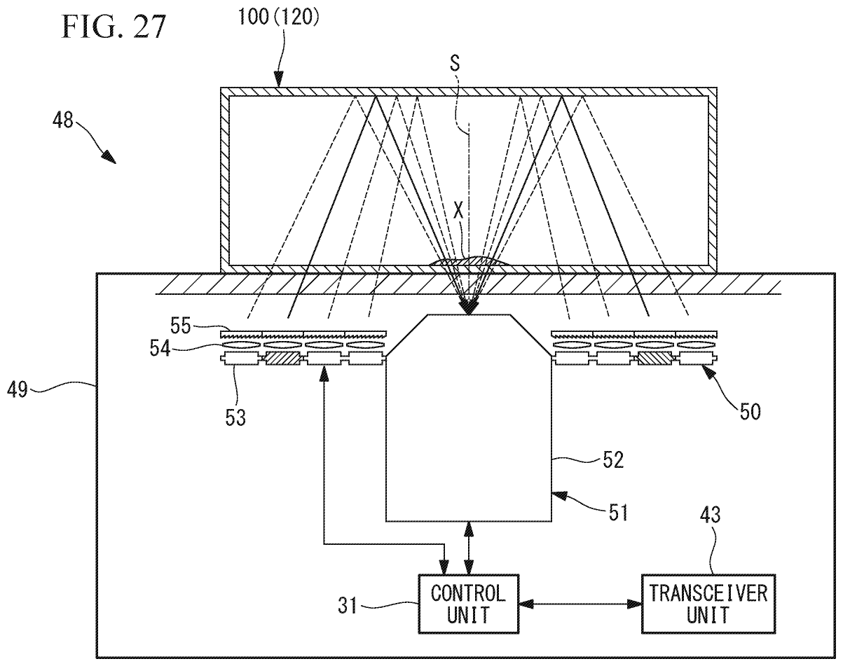

Furthermore, an observation device 48 shown in FIG. 27 may be adopted as the culture-state monitoring device 21.

For example, the observation device 48 includes a base 49 on which a culture container 100 or 120 accommodating a sample, such as the cells X, together with a medium is mounted, a light source unit 50 provided in the base 49, an image capturing unit 51, a transceiver unit 43, and a control unit 31.

In this case, the control unit 31 may transmit a signal to the medium changing device 1, 12, or 15 via the transceiver unit 43 when the state of the cells X as observed by using the observation device 48 has become a predetermined state, and the medium changing device 1, 12, or 15 that has received the signal may start discharging and supplying the medium by using the signal as a trigger. For example, the predetermined state refers to a predetermined number of cells, a predetermined cell density, a predetermined area occupied by the cells X, a predetermined form of cells, or the like.

The culture container 100 or 120 is, for example, a cell culture flask having a top plate, and is formed of an optically transparent material.

The base 49 is, for example, a casing, and the light source unit 50, the image capturing unit 51, the transceiver unit 43, and the control unit 31 are included inside the casing. At least a portion of the top face of the base 49 has a mounting face made of an optically transparent material, such as a glass, and the culture container 100 or 120 is mounted on this mounting face.

Since the humidity is high in an incubator, it is preferable that the base 49 has a waterproof structure. The image capturing unit 51 is disposed under the mounting face inside the base 49. The image capturing unit 51 includes an objective lens 52 that collects light coming from above and passing through the mounting face of the base 49 and a photographing optical system (not shown) that photographs the light transmitted through the cells X. The light source unit 50 is disposed outward in the radial direction of the objective lens 52, and radiates illumination light upward through the mounting face of the base 49.

The light source unit 50 includes a plurality of LED light sources (light sources) 53 disposed with gaps in the circumferential direction and in the radial direction, a plurality of collimator lenses 54 that are disposed in association with the individual LED light sources 53 and that substantially collimate the illumination light generated by the individual LED light sources 53, and a diffuser plate 55 that diffuses the illumination light collimated by the collimator lenses 54.

The light source unit 50 is capable of independently turning on specific LED light sources 53. FIG. 27 indicates LED light sources 53 that are turned on by hatching. The illumination light emitted from the LED light sources 53 is transmitted through the mounting face of the base 49 and the bottom face of the culture container 100 or 120 from the lower side to the upper side, is then reflected on the inner face of the top plate of the culture container 100 or 120, is then transmitted from obliquely above through the cells X, the bottom face of the culture container 100 or 120, and the mounting face of the base 49, and is thus introduced to the objective lens 52.

By turning on only LED light sources 53 at different positions in the radial direction of the objective lens 52, it is possible to switch the angles of illumination light indicated by solid lines to the angles of illumination light indicated by broken lines in FIG. 27.

Alternatively, by turning on only LED light sources 53 at specific positions in the circumferential direction of the objective lens 52, it is possible to illuminate the cells X only from specific directions in the circumferential direction. Alternatively, by turning on LED light sources 53 disposed in two or more directions in the circumferential direction of the objective lens 52, in particular, directions axially symmetric to the optical axis of the objective lens 52, it is possible to irradiate the cells X with illumination light in which illumination variations are reduced.

Alternatively, the light source unit 50 may include a plurality of LED light sources (light sources) 53 disposed around the objective lens 52 with gaps only in the circumferential direction, a plurality of collimator lens 54 that are disposed in association with the individual LED light sources 53 and that substantially collimate illumination light generated by the individual LED light sources 53, and a diffuser plate that diffuses the illumination light collimated by the collimator lenses 54.

Alternatively, for the LED light sources (light sources) 53, four collimator lenses 54, and four diffuser plates 55 may be disposed with 90° gaps in the circumferential direction.

An observation method using the observation device 48 according to this embodiment, configured as described above, will be described below.

In order to observe cells X by using the observation device 48 according to this embodiment, as shown in FIG. 27, the cells X are accommodated in the culture container 100 or 120, and then the culture container 100 or 120 is mounted on the mounting face of the base 49 at a position where the bottom face thereof is located on the lower side, with the base 49 bonded to the bottom face of the culture container 100 or 120.

Then, in this state, illumination light is generated by activating one of the LED light sources 53 of the light source unit 50. The illumination light generated by the LED light source 53 is collimated by the collimator lens 54 disposed in association with the LED light source 53 and is diffused by the diffuser plate 55, and the resulting illumination light is transmitted through the mounting face of the base 49 and the bottom face of the culture container 100 or 120 from the lower side to the upper side (emitting step), and is reflected on the inner face of the top plate of the culture container 100 or 120 and irradiates the cells X from obliquely above (reflecting step).

Of the illumination light irradiating the cells X, transmitted light of the illumination light, transmitted through the cells X, is transmitted through the bottom face of the culture container 100 or 120 and the mounting face of the base 49 from the upper side to the lower side and is introduced to the objective lens 52 (transmitting step). At this time, the illumination light is refracted or scattered depending on the shape and refractive index of the cells X or is dimmed according to the transmittance of the cells X, thereby becoming transmitted light carrying information about the cells X, the transmitted light is collected by the objective lens 52, and the collected light is photographed by an image capturing element, which is not shown, of the image capturing unit 51 (photographing step).

With the observation device 48 according to this embodiment, since a photographing optical system including the light source unit 50 and the objective lens 52 is disposed under the cells X, an advantage is afforded in that the light source unit 50 and the photographing optical system are aggregated only on one side of the cells X, which makes it possible to reduce the thickness of the device. Furthermore, also with the observation device 48 having the reduced thickness, an advantage is afforded in that it is possible to observe an object, such as the cells X, by photographing transmitted light, without having to mark the object.

Furthermore, since the illumination light from the light source unit 50 is emitted from outside in the radial direction of the objective lens 52 and is then reflected on the inner face of the top plate of the culture container 100 or 120, whereby the illumination light irradiates the cells X from obliquely above and is collected by the objective lens 52, it is possible to form bright and dark regions in an image of the cells X by suitably setting the incidence angle on the cells X. Therefore, an advantage is afforded in that it is possible to acquire an easily viewable image even with a transparent object, such as the cells X.

Furthermore, in this embodiment, since the light source unit 50 includes the plurality of LED light sources 53 that are arrayed in the radial direction around the objective lens 52 and that can be turned on independently of each other, it is possible to change the irradiation angle of the illumination light incident on the cells X by changing the radial-direction position of the LED light source 53 that is turned on, as indicated by broken lines in FIG. 27. This makes it possible to provide illumination that creates a bright field of view with little illumination variations in the case where the incident angle is smaller than the pickup angle of the objective lens 52. Meanwhile, it is possible to provide illumination that creates a dark field of view in which microstructures are emphasized in the case where the incident angle is greater than the pickup angle of the objective lens 52. Furthermore, it is possible to provide oblique illumination that enables stereoscopic viewing of the cells X in the case where the incident angle is equivalent to the pickup angle of the objective lens 52.

Furthermore, in this embodiment, since the light source unit 50 includes the plurality of LED light sources 53 that are arrayed in the circumferential direction around the objective lens 52 and that can be turned on independently of each other, it is possible to change the irradiation angle of the illumination light incident on the cells X by changing the circumferential-direction position of the LED light source 53 that is turned on. This makes it possible to change the direction of shading in a formed image of the cells X, thereby changing the appearance thereof.

Furthermore, by simultaneously turning on a plurality of LED light sources 53 at different positions in the circumferential direction, in particular, by simultaneously turning on a plurality of LED light sources 53 disposed in axial symmetry, an advantage is afforded in that illumination variations are reduced, which makes it possible to acquire an image of the cells X with little variations.

Furthermore, in this embodiment, since the diffuser plates 55 are provided in association with the individual LED light sources 53, the illumination light emitted from the LED light sources 53 is diffused uniformly, which makes it possible to irradiate the cells X with illumination light having a uniform intensity with little illumination variations.

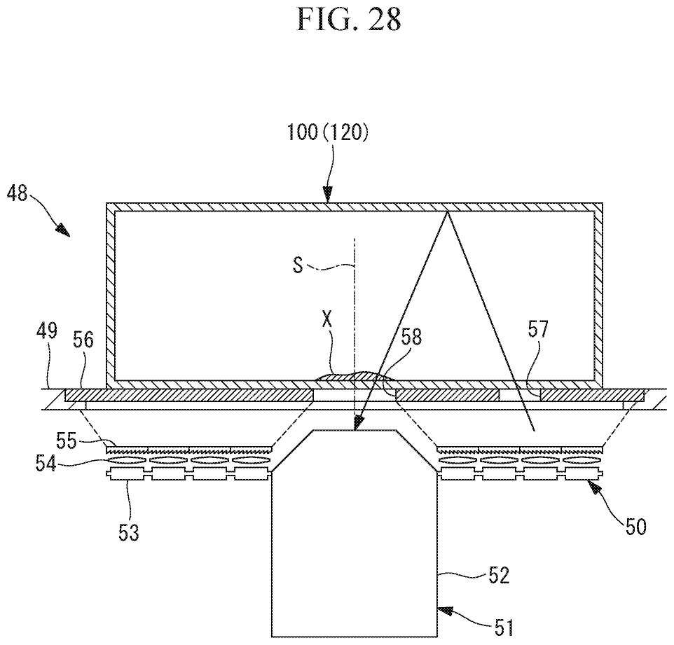

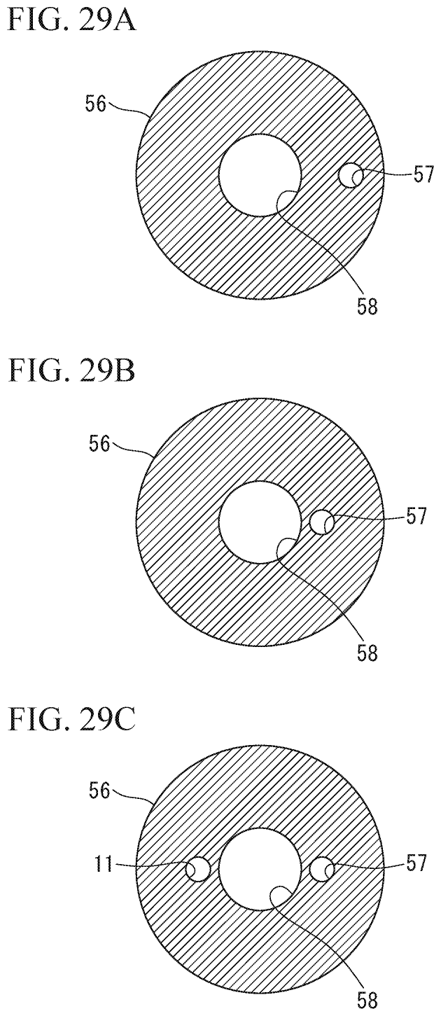

In this embodiment, the plurality of LED light sources 53 are disposed in the form of an array and are turned on independently of each other to switch the irradiation angle, irradiation direction, or the like of the illumination light. Alternatively, as shown in FIGS. 28, 29A, 29B, 29C, 30A, and 30B, the light source unit 50 may include LED light sources 53 disposed around the objective lens 52 and a light blocking member 56 that is disposed above the LED light sources 53 to block the illumination light from the LED light sources 53.

More specifically, the light blocking member 56 is provided with an opening 57 that opens at a portion in the circumferential direction or the radial direction thereof and a transmission hole through which light reflected on the inner face of the top plate of the culture container 100 or 120 and transmitted through the cells X is transmitted. Thus, by replacing the light blocking member 56, it is possible to adjust the position of the opening 57 so as to change the irradiation angle or irradiation direction of the illumination light. In this case, although the light source unit 50 may include LED light sources 53, collimator lenses 54, and diffuser plates 55, similarly to the configuration described above, there is no need for a function of switching the position at which illumination light is emitted, and a light source unit including an arbitrary light source that is capable of emitting illumination light from a range wider than the opening 57 may be adopted.

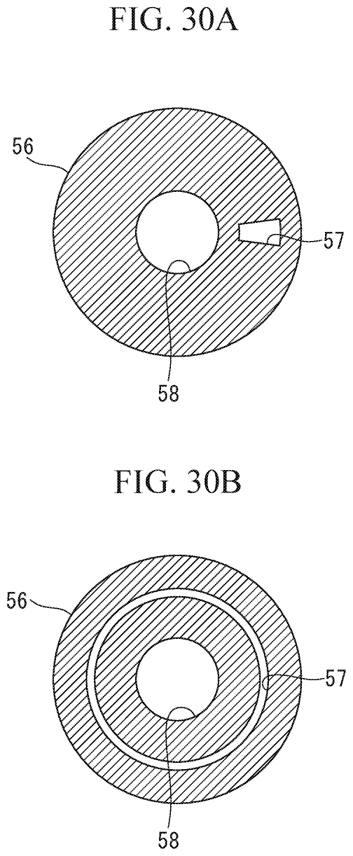

FIGS. 29A, 29B, and 29C show examples in which the opening 57 has a circular shape and the radial-direction positions of the openings 57 and number of the openings 57 vary. FIG. 30A shows a case where the opening 57 has a fan shape, and FIG. 30B shows a case where the opening 57 has a ring shape. The size, position, and shape of the opening 57 may be chosen arbitrarily.

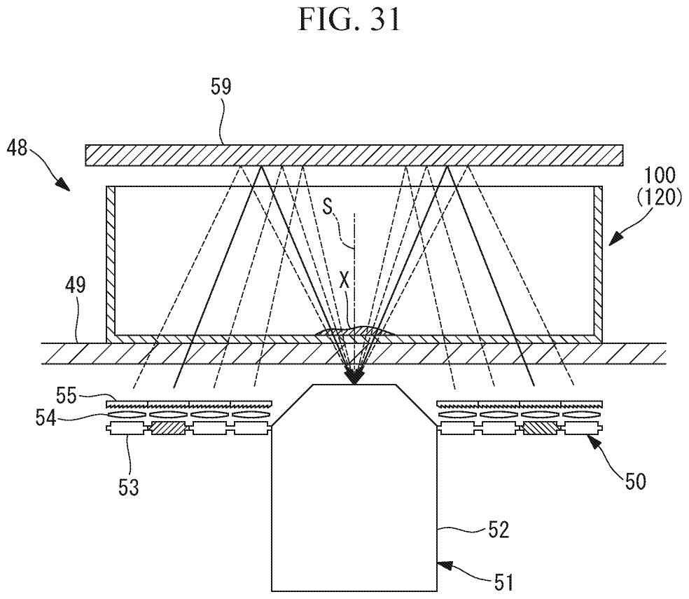

Furthermore, in this embodiment, the cells X are accommodated in the culture container 100 or 120 having a top plate, such as a cell culture flask, and illumination light is reflected on the inner face of the top plate of the culture container 100 or 120; however, there is no limitation to this configuration. For example, in the case where the cells X are accommodated in a culture container 100 or 120 not having a top plate, such as a petri dish not having a lid, as shown in FIG. 31, a reflecting member 59, such as a mirror, may disposed at a position where the top opening of the petri dish is closed therewith so that light transmitted through the bottom face of the culture container 100 or 120 from the lower side to the upper side will be reflected by the reflecting member 59. The reflecting member 59 may be provided such that the reflecting member 59 can be placed at and withdrawn from a position above the cells X by linear movement or pivotal movement.

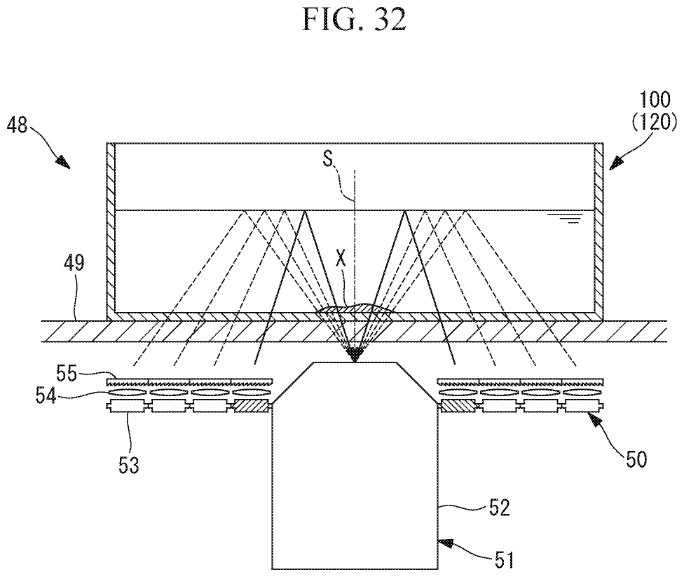

Alternatively, in the case where the cells X are accommodated in a culture container 100 or 120 not having a top plate, such as a petri dish, as shown in FIG. 32, a solution such as a culture medium or a phosphate buffer solution may be supplied into the culture container 100 or 120, immersing the cells X in the solution. Then, illumination light transmitted through the culture container 100 or 120 from the lower side to the upper side may be reflected by the top liquid surface of the solution. Also, in the case where the cells X are accommodated in a culture container 100 or 120 having a top plate, a solution such as a culture medium or a phosphate buffer solution may be supplied into the culture container 100 or 120, immersing the cells X in the solution.

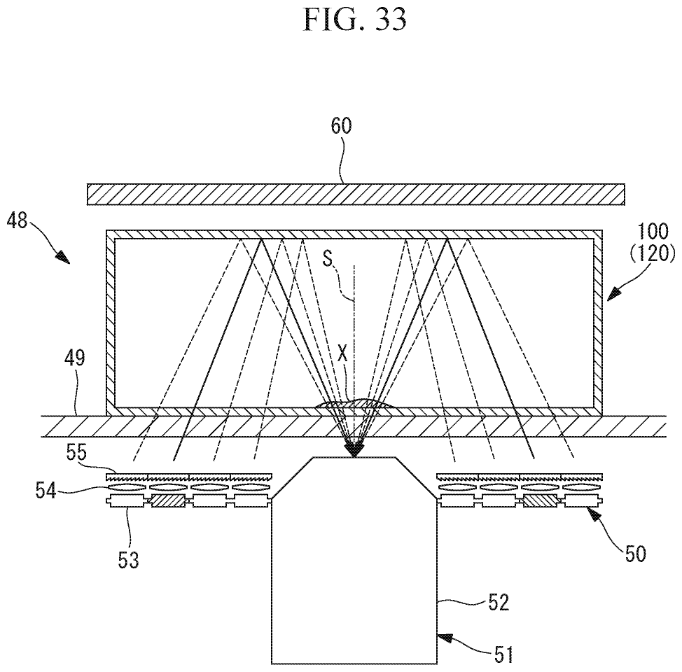

Furthermore, in this embodiment, as shown in FIG. 33, a light blocking member 60 formed of a material that blocks light may be provided above the top plate of the culture container 100 or 120 having a top plate.

With this configuration, external light from the outside is blocked by the light blocking member 60. This hinders external light from being introduced into the culture container 100 or 120 through the top plate of the culture container 100 or 120, which enables efficient observation.

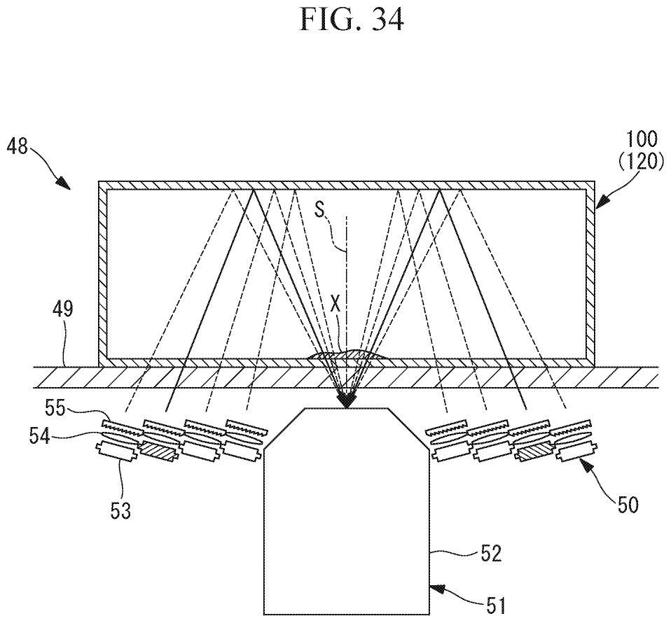

Furthermore, although the LED light sources 53, the collimator lenses 54, and the diffuser plates 55 are disposed substantially horizontally at positions along the mounting face of the base 49 in the example of the light source unit 50 in this embodiment, alternatively, as shown in FIG. 34, the LED light sources 53, the collimator lenses 54, and the diffuser plates 55 may be disposed so as to be slanted toward the optical axis S.

With this configuration, loss of the illumination light emitted from the LED light sources 53 is suppressed, which makes it possible to efficiently irradiate the cells X with the illumination light. Furthermore, although the light source unit 50 includes the diffuser plates 55 in the example given in this embodiment, the diffuser plates 55 may be omitted.

In the example given in this embodiment, the observation device 48 serving as a culture-state monitoring device includes the transceiver unit 43 and the control unit 31 serving as the control device 23. Alternatively, the transceiver unit 43 and the control unit 31 may be omitted from the observation device 48, and the culture system 20 may include the control device 23 separately from the observation device 48.

The transceiver 43 sends information to and receives information from the external control device 44 installed outside an incubator, in a wired or wireless manner. The transceiver unit 43 sends an image acquired by the image capturing unit 51 to the external control device 44 outside in a wired or wireless manner, and receives information from the external control device 44 and sends the information to the control unit 31.

The control unit 31 activates the light source unit 50, the image capturing unit 51, and the transceiver unit 43 on the basis of the information from the external control device 44. Alternatively, for example, the control unit 31 includes a timer, which is not shown, and periodically activates the light source unit 50, the image capturing unit 51, and the transceiver unit 43.

The external control device 44 is disposed outside an incubator, and sends information to and receives information from the observation device 48 in the incubator in a wired or wireless manner. Furthermore, the external control device 44 sends information to and receives information from a user terminal, which is not shown, in a wired or wireless manner.

In this case, the external control device 44 receives sample data, such as an image, sent from the observation device 48, and sends the sample data to the user terminal. Furthermore, the external control device 44 sends information to the observation device 48 in the incubator on the basis of the information sent from the user terminal.

The external control device 44 may include a display means (monitor), which is not shown, and may display the sample data sent from the observation device 48 on the display means. In this case, the user terminal may be omitted.

The external control device 44 may include an input means such as a keyboard or a mouse, which is not shown, and may send information input via the input means to the observation device 48 in the incubator. In this case, the user terminal may be omitted.

The user terminal includes a display unit and an input unit, and sends information to and receives information from the external control device 44 in a wireless manner.

The user terminal receives the sample data sent from the external control device 44 and displays the sample data on the display unit thereof. Furthermore, the user terminal sends information input via the input unit thereof to the external control device 44. The user terminal is, for example a PC, a smartphone, or a tablet.

Furthermore, a modification of the first embodiment described above will be described below with reference to the drawings.

In the description of this embodiment, parts that have the same configurations as those in the medium changing device 1 according to the first embodiment described above will be denoted by the same reference signs, and descriptions thereof will be omitted.

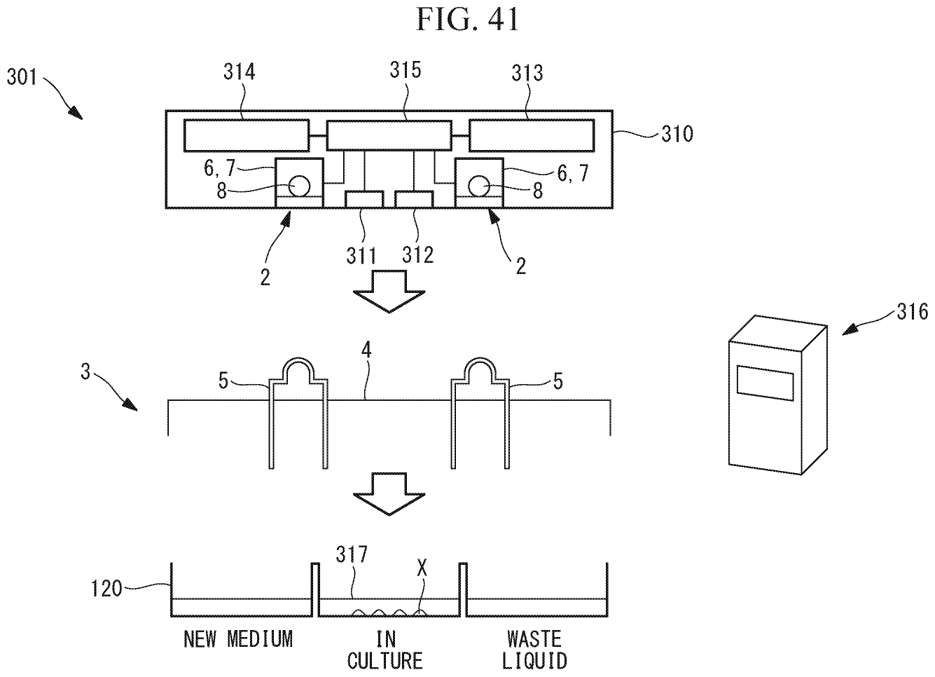

As shown in FIG. 41, a medium changing system (culture system) 301 according to this embodiment includes a casing 310, an illumination unit 311, a light receiving unit 312, a battery unit 313, an external communication unit 314, a control unit 315, and an external control unit 316.

The casing 310 accommodates power units 2, the illumination unit 311, the light receiving unit 312, the battery unit 313, the external communication unit 314, and the control unit 315. Furthermore, a transparent window that allows light radiated by the illumination unit 311 and light received by the light receiving unit 312 to be transmitted therethrough is provided in a portion of the casing 310. Furthermore, the casing 310 can be attached to and detached from a liquid transfer unit 3 in which tubes 5 are provided through a lid member 4.

The illumination unit 311 radiates light having a plurality of colors toward a medium 317 on which cells X are cultured.

For example, the light radiated from the illumination unit 311 includes at least three colors, such as red light, green light, and blue light. The illumination unit 311 may be a white light source or the like that is capable of simultaneously radiating light having a plurality of colors, or may be formed by providing a plurality of monochrome light sources or the like that are capable of radiating light in individual colors independently of each other. Alternatively, the illumination unit 311 may be configured to be capable of radiating light having a plurality of colors by combining a multi-color light source and a monochrome light source.

The light receiving unit 312 is capable of receiving light having a plurality of colors, radiated from the illumination unit 311 and transmitted through the medium 317, and detecting the intensities of light having the individual colors independently of each other.

For example, in the case where the illumination unit 311 is a white light source that emits white light including light having at least three colors, the light receiving unit 312 is formed of colors sensors that are capable of detecting the intensities of light having at least three colors independently of each other. Alternatively, the light receiving unit 312 may be formed of monochrome sensors provided with optical filters that pass light having specific colors and may detect the intensities of light having at least three colors independently of each other.

Alternatively, in the case where the illumination unit 311 is formed of a plurality of monochrome light sources, the light receiving unit 312 is formed of color sensors that are capable of detecting the intensities of light having at least three colors independently of each other. Alternatively, in the case where the illumination unit 311 is formed of a plurality of monochrome light sources and monochrome light is radiated sequentially and independently, the light receiving unit 312 is formed of a monochrome sensor and may detect the intensities of monochrome light sequentially radiated from the illumination unit 311.

The battery unit 313 supplies electric power to the illumination unit 311, the light receiving unit 312, the external communication unit 314, and the control unit 315. The battery unit 313 may be, for example, a replaceable battery or a chargeable battery built into the medium changing system 301. Alternatively, the battery unit 313 may be connected to a power supply provided outside the casing 310 in order to supply electric power.

The external communication unit 314 is electrically connected to the control unit 315, and sends and receives information between the external control unit 316 and the control unit 315. The external communication unit 314 may be connected to the external control unit 316 in a wired manner or in a wireless manner.

The control unit 315 includes, for example, a CPU and a memory. The control unit 315 controls the operations of the individual components of the medium changing system 301 as the CPU executes various programs stored in the memory.

For example, the control unit 315 controls the radiation of light by the illumination unit 311, the detection of light by the light receiving unit 312, the operations of the power units 2, etc. Furthermore, the control unit 315 sends the intensities of light received by the light receiving unit 312 to the external control unit 316 via the external communication unit 314, and controls the operations of the individual components of the medium changing system 301 on the basis of information received from the external control unit 316.

An example of the external control device 316 is a personal computer (PC).

For example, the control functionality may be realized by a PC including a CPU and a memory, in which the CPU executes a control program stored in the memory.

For example, the external control unit 316 may calculate an environment index value of the medium 317 on the basis of the light intensities received from the medium changing system 301, and may determine that it is necessary to change the medium 317 and instructs the medium changing system 301 to change the medium 317 in the case where the calculated environment index value is less than a predetermined threshold. In this case, in the medium changing system 301, the control unit 315 changes the medium 317 by controlling the operations of the power units 2 on the basis of the instruction for changing the medium 317 from the external control unit 316.

Alternatively, the external control unit 316 may calculate an environment index value of the medium 317 on the basis of the light intensities received from the medium changing system 301 and may provide a user with the environment index value of the medium 317 or information indicating the environment index value. Then, the external control unit 316 may transmit an operation instruction to the medium changing system 301 on the basis of an input from the user. In this case, in the medium changing system 301, the control unit 315 changes the medium 317 by controlling the operations of the power units 2 of the medium changing system 301 on the basis of an instruction from the external control unit 316.

Furthermore, the external control unit 316 may include another memory to accumulate and store information about the light intensities detected by the light receiving unit 312 or information about the environment index value.

Furthermore, alternatively, the control unit 315 may calculate an environment index value of the medium 317 on the basis of the light intensities detected by the light receiving unit 312, and may determine that it is necessary to change the medium 317 and change the medium 317 by controlling the operations of the power units 2 in the case where the calculated environment index value is less than a predetermined threshold. In this case, there is no need to configure the external control unit 316, and the external control unit 316 may be omitted.

The environment index value is, for example, the PH value of the medium 317 or a parameter such as the number of cells or the culture period.

With this configuration, since the medium changing function and the culture-environment monitoring function are configured in an integrated fashion, it is possible to simplify the system as a whole. Therefore, it is possible to reduce the size of the system as a whole compared with the case where a device having the medium changing function and a device having the culture-environment monitoring function are provided separately.

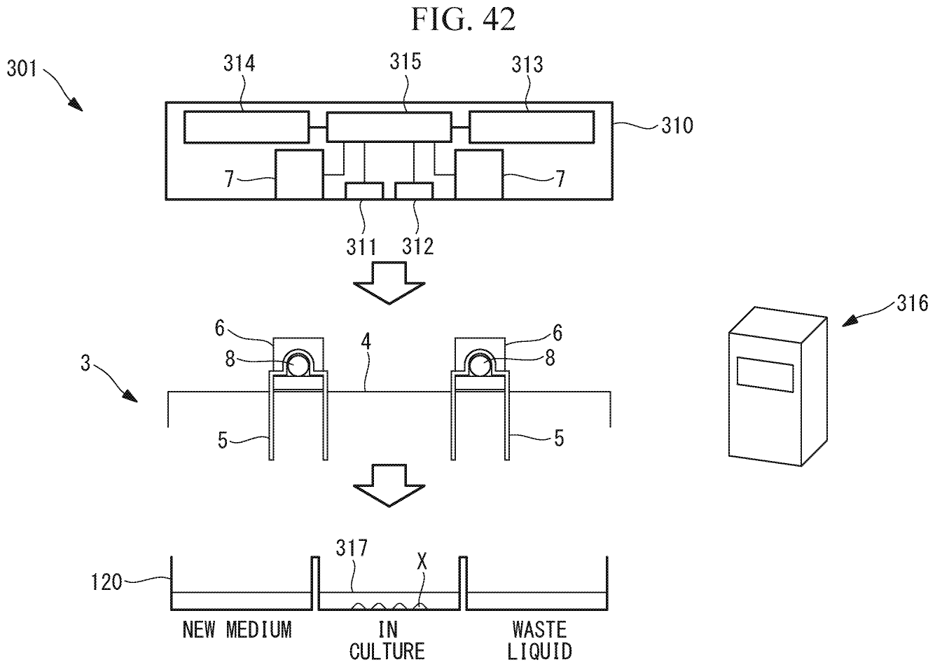

In this modification, for example, as shown in FIG. 42, pump bodies including rotors 8 may be constructed together with the liquid transfer unit 3. In this case, the liquid transfer unit 3 and the pump bodies are mounted on the driving units 7 of the casing 310, which makes it possible to transfer liquid. Alternatively, the casing 310 may be constructed together with the liquid transfer unit 3.

Furthermore, a reflecting member having high reflectivity may be disposed on the path of light radiated from the illumination unit 311 and transmitted through the medium 317 and the culture container 120. In other words, the culture container 120 and the medium changing system 301 may be disposed in this order on the reflecting member. Since a large number of through holes are provided in a rack of an ordinary incubator, in the case where the medium changing system 301 is disposed on a rack of an incubator, the intensity of reflected light and the intensity of light detected by the light receiving unit 312 vary depending on the position where the medium changing system 301 is disposed. However, by disposing the reflecting member, it is possible to suppress variations in the light intensity depending on the place where the medium changing system 301 is disposed, thereby attaining a uniform light intensity.

Although the use for a single culture region is given as an example in this embodiment, medium changes in a plurality of culture regions may be allowed by a single medium changing system. In the case where medium changes in a plurality of regions are performed, a driving unit 2 may be used commonly for the medium changes in the plurality of regions, a plurality of illumination units 311 and a plurality of light receiving units 312 may be provided, and the illumination units 311 and the light receiving units 312 may be provided individually for the plurality of culture regions. In this case, the media 317 may be changed on the basis of information indicating the environment index value for one of the plurality of culture regions, or the media 317 may be changed on the basis of the average of the environment index values for the plurality of culture regions.

The above-described embodiment also leads to the following invention.

The present invention, in one aspect thereof, provides a medium changing device including a flat-plate-shaped lid member disposed at a position where two or more regions in which a medium may be stored are covered therewith, the regions being disposed adjacent to each other and being open upward; one or more flow-path members each disposed so as to penetrate the lid member in a thickness direction with openings at either end thereof exposed on one side of the lid member and with an intermediate section thereof exposed on the other side of the lid member, and each disposed at a position where one of the regions is bridged to another therewith when the lid member is disposed at the position where the regions are covered; and a pump that is disposed on the other side of the lid member and that acts on the intermediate sections of the flow-path members, exposed on the other side, so as to cause the medium to flow from the opening at one end toward the opening at the other end of each of the flow-path members.

According to this aspect, when the flat-plate-shaped lid member is disposed at the position where the two or more regions are covered therewith, both ends of each of the flow-path members penetrating the lid member in the thickness direction are disposed on one side of the lid member, and one end of the flow-path member is disposed in one region, while the other end of the flow-path member is disposed in another region, whereby the flow-path member is disposed at a position where the flow-path member bridges these two regions. In this state, the pump disposed on the other side of the lid member is driven, whereby the pump acts on the intermediate section of the flow-path member, which causes the medium to flow from the opening at the one end to the opening at the other end of the flow-path member.

That is, a new medium is stored in one region, cells are cultured in another region, and the pump is activated when a timing for medium change arrives. This makes it possible to supply the new medium to the region where the cells are being cultured.

In this case, since each of the flow-path members through which the medium is caused to flow is disposed along a short path that comes back after twice penetrating the flat-plate-shaped lid member in the thickness direction so that the pump can be caused to act thereon, it is possible to reduce the amount of medium remaining in the flow-path member. Therefore, the consumption of expensive media can be reduced, and it becomes possible to use general-purpose containers, which make it possible to reduce the cost of consumables.

In the above aspect, the pump may be provided removably from the lid member.

With this configuration, it is possible to configure the lid member and the flow-path members as disposable parts, while reusing the pump, which is expensive.

Furthermore, in the above aspect, the pump may include a pump body and a driving unit that drives the pump body, the pump body may be fixed to the lid member, and the driving unit may be removably attached to the pump body.

With this configuration, it is possible to attach and detach the driving unit while keeping the pump body and the flow-path members combined. This makes it possible to precisely transfer the liquid medium.

Furthermore, in the above aspect, the flow-path members may be formed only of tubes made of a flexible material, and the pump may be a peristaltic pump that transfers liquid by squeezing the flow-path members from radially outward.

With this configuration, it is possible to readily attach the pump to the outside of the tubes constituting the flow-path members, which makes it possible to transfer the liquid medium without having to bring the pump into contact with the medium. This facilitates the reuse of the pump.

Furthermore, the present invention, in another aspect thereof, provides a medium changing device including a flat-plate-shaped lid member disposed at a position where a region that is open upward and in which a medium may be stored is covered therewith; two or more flow-path members each penetrating the lid member in a thickness direction with openings at either end thereof disposed on either side of the lid member in the thickness direction; two or more containers each connected to one end of one of the flow-path members disposed thereabove when the lid member is disposed at the position where the region is covered; and two or more pumps that are disposed between the containers and the lid member and that act on intermediate sections of the flow-path members so as to cause the medium in the flow-path members to flow.

According to this aspect, when the flat-plate-shaped lid member is disposed at the position where the region in which a medium may be stored is covered therewith, one end of each of the two or more flow-path members penetrating the lid member in the thickness direction is connected to a corresponding one of different containers above the lid member, and the other end thereof is disposed in the same region. In this state, one of the pumps disposed between the containers and the lid member is driven, whereby the pump sucks the old medium in the region and discharges the old medium into the container, and the other pump is driven to cause a new medium in the container to flow toward the region.

That is, a new medium is stored in one container, a space in which an old medium may be stored is formed in another container, cells are cultured in the region, and the pumps are activated when a timing for medium change arrives. This makes it possible to suck the old medium from the region where the cells are being cultured and to supply the new medium.

In this case, since each of the flow-path members through which the medium is caused to flow is disposed along a relatively short path penetrating the flat-plate-shaped lid member in the thickness direction and connected to a container via a pump, it is possible to reduce the amount of medium remaining in the flow-path member. Therefore, the consumption of expensive media can be reduced, and it becomes possible to use general-purpose containers, which make it possible to reduce the cost of consumables.

In the above aspect, the pumps may be provided removably from the lid member.

With this configuration, it is possible to configure the lid member and the flow-path members as disposable parts, while reusing the pumps, which are expensive.

Furthermore, in the above aspect, each of the pumps may include a pump body and a driving unit that drives the pump body, the pump body may be fixed to the lid member, and the driving unit may be removably attached to the pump body.

With this configuration, it is possible to attach and detach the driving unit while keeping the pump bodies and the flow-path members combined. This makes it possible to precisely transfer the liquid medium.

Furthermore, in the above aspect, the flow-path members may be formed only of tubes made of a flexible material, and the pumps may be peristaltic pumps that transfer liquid by squeezing the flow-path members from radially outward.

With this configuration, it is possible to readily attach the pumps to the outside of the tubes constituting the flow-path members, which makes it possible to transfer the liquid medium without having to bring the pumps into contact with the medium. This facilitates the reuse of the pumps.

Furthermore, the present invention, in another aspect thereof, provides a medium changing device including a flat-plate-shaped lid member disposed at a position where a region that is open upward and in which a medium may be stored is covered therewith; two or more flow-path members each penetrating the lid member in a thickness direction with openings at either end thereof disposed on either side of the lid member in the thickness direction; two or more containers each connected to one end of one of the flow-path members disposed thereabove when the lid member is disposed at the position where the region is covered; and two or more valves that are disposed at intermediate sections of the flow-path members between the containers and the lid member and that releasably close flow paths inside the flow-path members, wherein the pressure inside at least one of the containers is lowered.

According to this aspect, when the flat-plate-shaped lid member is disposed at the position where the region in which a medium may be stored is covered therewith, one end of each of the two or more flow-path members penetrating the lid member in the thickness direction is connected to a corresponding one of different containers above the lid member, and the other end thereof is disposed in the same region. In this state, the valve disposed between the container having the lowered interior pressure and the lid member is released, whereby the old medium in the region is sucked and is discharged into the container. Furthermore, the valve disposed between the container storing a new medium and the lid member is released, which causes the new medium in the container to flow toward the region.

That is, a new medium is stored in one container, a space in which an old medium may be stored is formed in another container, which has a lowered pressure, cells are cultured in the region, and the valves are released when a timing for medium change arrives. This makes it possible to suck the old medium from the region where the cells are being cultured and to supply the new medium to the region.

In this case, since each of the flow-path members through which the medium is caused to flow is disposed along a relatively short path penetrating the flat-plate-shaped lid member in the thickness direction and connected to a container via a pump, it is possible to reduce the amount of medium remaining in the flow-path member. Therefore, the consumption of expensive media can be reduced, and it becomes possible to use general-purpose containers, which make it possible to reduce the cost of consumables. Furthermore, since no driving source is required in order to suck and supply media, the structure is simplified.

In the above aspect, the valves may include partitions that close the flow paths inside the flow-path members and that can be broken by applying an external force.

With this configuration, it is possible to keep the valves closed in a state where the partitions are not broken. Then, it is possible to readily suck and supply media by releasing the valves just by breaking the partitions with an external force. This makes is possible to further simplify the structure, thereby reducing cost.

Furthermore, the present invention, in another aspect thereof, provides a culture system including one of the medium changing devices described above; a culture-state monitoring device that monitors the state in the region or regions; and a control device that controls the pump in accordance with the state in the region or region as detected by the culture-state monitoring device.

According to this aspect, the culture-state monitoring device monitors the state in the region or regions, and when it is determined that a timing for medium change has arrived, it is possible to perform medium change by controlling the pumps by means of the control device. This makes it possible to change the medium without having to take out the containers from an incubator.

In the above aspect, when the culture-state monitoring device has detected that the state in the region or one of the regions is suitable for medium change, the control device may control the pump so as to discharge the medium in the region detected as being suitable for medium change from that region and so as to supply a new medium to the region from which the medium has been discharged.

Furthermore, the present invention, in another aspect thereof, provides a culture system including one of the medium changing devices described above; a culture-state monitoring device that monitors the state in the region; and a control device that releases the valves in accordance with the state in the region as detected by the culture-state monitoring device.

In the above aspect, when the culture-state monitoring device has detected that the state in the region is suitable for medium change, the control device may control the valves so as to release the valve for the flow-path member connected to the container having the lowered pressure, thereby causing the medium in the region to be sucked into that container, and so as to release, after completion of the suction, the valve for the flow-path member connected to another one of the containers, which stores a new medium, thereby supplying the new medium in that container into the region.

Furthermore, in the above aspect, the culture-state monitoring device may monitor the color of the medium.

Furthermore, in the above aspect, the culture-state monitoring device may monitor the number of cells being monitored in the region or regions.

Furthermore, in the above aspect, the culture system may further include a casing that accommodates the medium changing device and the culture-state monitoring device together.

### REFERENCE SIGNS LIST

1, 12, 15, 200 Medium changing device

2, 202 Power unit (pump)

4, 204 Lid member

5, 5a, 5b, 205 Tube (flow-path member)

6, 206 Pump body

7, 207 Driving unit

14a, 14b Tank (container)

16 Valve

20 Culture system

21 Culture-state monitoring device

23 Control device

44, 316 External control device (control device)

48 Observation device (culture-state monitoring device)

110 Well (region)

120 Culture dish (culture container, region)

301 Medium changing system (culture system)

X Cells

1. A medium changing device comprising:
a flat-plate-shaped lid disposed at a position where two or more regions in which a medium may be stored are covered therewith, the regions being disposed adjacent to each other and being open upward;
one or more flow-path members each disposed so as to penetrate the lid in a thickness direction with openings at either end thereof exposed on one side of the lid and with an intermediate section thereof exposed on the other side of the lid, and each disposed at a position where one of the regions is bridged to another therewith when the lid is disposed at the position where the regions are covered; and
a pump that is disposed on the other side of the lid and that acts on the intermediate sections of the flow-path members, exposed on the other side, so as to cause the medium to flow from the opening at one end toward the opening at the other end of each of the flow-path members.

2. The medium changing device according to claim 1, wherein the pump is provided removably from the lid.

3. The medium changing device according to claim 1,
wherein the pump includes a pump body and a motor that drives the pump body,
wherein the pump body is fixed to the lid, and
wherein the motor is removably attached to the pump body.

4. The medium changing device according to claim 1,
wherein the flow-path members are formed of tubes, and
wherein the pump is a peristaltic pump that transfers liquid by squeezing the flow-path members from radially outward.

5. A culture system comprising:
the medium changing device according to claim 1;
a culture-state monitoring device that monitors and detects a state in the regions; and
a controller that controls the pump in accordance with the state in the regions as detected by the culture-state monitoring device.

6. The culture system according to claim 5, wherein:
the culture-state monitoring device detects the state in the regions; and
the controller controls the pump, discharges the medium in the regions, and supplies a new medium to the regions.

7. The culture system according to claim 5, wherein each of the regions is a culture region where cells are being cultured.

8. The culture system according to claim 5, wherein the culture-state monitoring device includes:
an irradiation optical system that irradiates with light in each of the regions; and
a sensor that detects light in each of the regions.

9. The culture system according to claim 8, wherein the light by the irradiation optical system is radiated in a horizontal direction from a lateral side of each of the regions.

10. The culture system according to claim 8, wherein the culture-state monitoring device includes a driving means for moving the irradiation optical system or the sensor.

11. The culture system according to claim 5, wherein an instruction for changing the medium in each of the regions is an intensity of light detected by the culture-state monitoring device.

12. The culture system according to claim 5, wherein the controller calculates a level of light absorption by the medium by using an intensity of light detected by the culture-state monitoring device.

13. The culture system according to claim 5, wherein the culture-state monitoring device monitors a color of the medium.

14. The culture system according to claim 5, wherein the controller is provided outside an incubator.

15. The culture system according to claim 5, further comprising a casing that accommodates the medium changing device and the culture-state monitoring device together.

16. The culture system according to claim 15, wherein the casing has a waterproof structure.

17. The culture system according to claim 5, wherein the culture-state monitoring device has a function for observing cells.

18. The culture system according to claim 17, wherein the culture-state monitoring device monitors the number of cells in each of the regions.

19. The culture system according to claim 17, wherein the culture-state monitoring device causes light from a light source to be reflected by a reflecting member and performs observation by irradiating the cells in each of the regions with the reflected light.

20. The culture system according to claim 19, wherein the reflecting member is a top plate of a culture container constituting each of the regions.
